SEQUENCE LISTING (60 sequences)

SEQ ID NO: 1 - DNA, 401 bp, Canis familiaris
  misc_feature (39)..(39): n = A, G, C or T
ctggagacccaggattgagtcccacatcgggttccctgnatggagcntgcttctccctctgcctctgtctctgcttttctctctctctctctctcatgaataaataaataaaatcttaaaaaaaaaaaaaaaagaaagaaatcctaacncccagtacctcagaatctgaccttatttggagattggatcttcacagagatratcaagttaaaacgacatcattcagacagatcctaatccaaaatganngacatctttacaaaaaaggaaatttggacattagagatatacctacaaggaaaatgatgggaagagacaccaatcatcgatggcgtctcccaagatgatgggagaagccaatcatctccaagccaagagaaagacctagaacagattctccc

SEQ ID NO: 2 - DNA, 401 bp, Canis familiaris
gtgatgtcaatgacagcccacagggccgtacagtatccatcctgaataactataaatggcagctcagatttgtcttcatcttttacttcacctcttcctctaggaagatgttcagtacccaatgtacttgccttggctaacctccccctcggtctacaccttagggtttagaaaggtagatctcagtggaatcttgcatgmagcaccacttcttatcttctctgccaccatctatcccaacaattaatgtgataaccagttttgtacaaaaatatttgacaggatttttgcaggcactgtacagtgctttcccctggatgacacatcatgattttggcattttccatacaattctctggtgttgtgaaatgagatacctgtacaaacctgctcaacactgg

SEQ ID NO: 3 - DNA, 401 bp, Canis familiaris
  misc_feature (54)..(54): n = A, G, C or T
gctacaaaagctccaggatatgctaaagaaacaaaaaagtaataaatacaaacnaatcaccttacttaaacctggactgtactgtgctgacatgattttgggtaaccagatatttaacaccttcaaatcagtgagctaagctgccaccattcgtctgaatttttgacacttactattctacaaagccttggaactcgggtsccatgcacaattcttcctttattcctgaacacctgctgttctgtctcctgtgtttcgatctttagtnccactgccctcttcccagttgatcccatcagaaacttttctgccccctttctctctctaatctcctccattggaatcagttactgaatcaaggagctctggcctgggtccntctcacagtccaccctttccag

SEQ ID NO: 4 - DNA, 401 bp, Canis familiaris
  misc_feature (45)..(45): n = A, G, C or T
aggaaccaggccgactcacccagtcttgactgtggggcggccagngcactggcatttcactgtgaagtgtgcagatgggatgatagagacttgcaagtggtacttgagtttagccttgaagaatgggtacaacctcaagaagagaaagtggaggaagcggtgttcctctcntagtaaacagcaagtgcaaaagtatggaaygttcaaggacagggaggagtgatgggatgtgatgaggtgtggggcatgtggtgagactgatgggaggtgatgctgaggctgggtgggcagaggatttgaggatatgctaaggagcggggtcttctagaaccccccagtggagatcccatgactttgttgtaggaacataaagttcagaccatgagtcaggagagcagagc

SEQ ID NO: 5 - DNA, 401 bp, Canis familiaris
ggtgctcagtgactgtggatcaagggagtgcctgaagtgaagcagagaaagttttgtggctaattgtctcctctcaggttgggatggcctgcagcttctggcagccactcccaggcacccagccagtgtttccagaccacggctggggctgctgtggatggtcctccagccacgcagtgatgaaaggcctcgtgaagaagraaatgaccttggtggttgctggatggaatgtttcccctggagaaatcacaaaatcaatcaggtgtccaggcctcagcctcactcctttcctgggccagcccagggtcctggggtcagctcctggggggaggtggcttcctctgacaggctagtttcctttccaggtctctgactctcctttgattttgtaatcctgttcc

SEQ ID NO: 6 - DNA, 401 bp, Canis familiaris
  misc_feature (161)..(161): n = A, G, C or T
aacacaggatcctccaagggatgagaaaagaaggggtcctaagggggtgacccctgggccagagcagccccacctgctcagtggcttaacagcttggactcctctgagctccagggtttctcatcaggaaaaacgaaatgagaaaactcaggtttacctanagggcgtggggagactaaaatgtgattacacacagggagygcctagcacttcaaaggggctcagtaagcctgcctgcccggctcccttgcccaccttctacgtggggggcgcggctgcggaggctctgcagcttcgggctcctaggggaggaacagggccgcnggaggccgccttctctccttagtgttattgaaaaatgttcaaaagaaacatttacacgaaaacaactttgggaaaac

SEQ ID NO: 7 - DNA, 401 bp, Canis familiaris
  misc_feature (86)..(86): n = A, G, C or T
ctccagattatagcgtggaggctacgacagtttctgtgtctctgggctcgctagaatgatgctcgataaaatctgtttcatctacntaataaggcgactctatccccgaggactatagggctgtatatacgtgtctatgaattgtcgcccagatgctggaaagctttatggaaattcctggacattgaccgctctcaaggstcccatggtaatttggagagtgtgacncttactctgctgtcctagcaaatatcaaagaaacgcgagtctggatgaaatgttattnacacgtgtccccgtcgggtgtgagtgtggagcaaaccagaggangaggcgtgtgcgtttgcagaaggaaggcanagacgggtgtgggacatgcccttgggaaaggtgtagatgcc

SEQ ID NO: 8 - DNA, 401 bp, Canis familiaris
tttataaatgattattatatatattaaacatatatatacattatatatataatagagtggcataaaatctgagtttgagttttaagatccttgaccattataattgcacatcctctatttatttctccaaaacttctacattgcccttggttagattccattttttcttctttaaattgcaccccagggattataggcacygaatgtgatctcataaacttctagaattataaaatatgcattagcaattgtaacacttggcattatgctagagtacagaaaatttgctatgtcctatccatcaacattacctcaaagagggtctgaagaatgcagcccaggtaattataaatgtttggagaatcagtaataatcatgagagtagatattaatgtattttc

SEQ ID NO: 9 - DNA, 401 bp, Canis familiaris
atgcataaggttattgccttagctgacttaaaattgcccccattcaatggtactatcaacctttagtgaagcccttaaaaaaacaaacaggttgaaaaagattaggagaggcaaatatagcatctgtctttagagctatcaactcaggaattctctcaattatgaaatcttgcagagaagttatttttcttttccaaattmtggtgatgacaatattccttactccagagctggcatttttacatcatctctgtcttgtgaaccatcatctgctccatctacttctgataaatctgcatcctcatcaccactcctgttgttcatcatctcagaggaatgatcaaaattagtatgtcttcaactgaatcatcttcccagtcgttccaattattattttttta

SEQ ID NO: 10 - DNA, 401 bp, Canis familiaris
  misc_feature (135)..(135): n = A, G, C or T
ttggtaattagacaaaagaagttaaattcagttcagacatttcatgcataaggtcctctgacaggcactggggatttgaagatgtattaaacataataagggtatgcatgcaggtggtgaagagggaagaggganagtttcagatggagggaataccatgtgtagagagcatcgtgagtggaaaatatatgggctgtgtgrcgtcaggtgaatggccagccacagctaaagggaatctgttcctccaaacaatataatctaagattgttgagtgtattgcacaaaccaagtctcgtgtcttatagatgatgtttgcaaattntagttagtacacacggatgcttaagtaaaactcatttgatgangggaaagcatttaaagccaaacaaaagcaaaaatat

SEQ ID NO: 11 - DNA, 401 bp, Canis familiaris
caaaagactcatcttgacttgaatttgacttagatggtgagattttggacattgagttctgtctgatgaggtttttgagggggtcttagacaaacattcaaaattgaaatgcattttgagttttaagggatggttcagtctggcagcattataagaatttaattgactaaatgtctccccttactgaaagaaggccaaaaygtttgctttgtaaggaagggtcatcaaatatatgtgttggttttatattatataagatttcaattaagttttacatcatctatgttcctaattttttttttatgtgggaggaatgtgaataacttattgccactgtgtaaaccatgactcatttataacagcctctgaattccttgactcttcccagcaaaaggtaagga

SEQ ID NO: 12 - DNA, 401 bp, Canis familiaris
ctgtgagaccgtggcccagtcagggctgcatcactgtgccttaatctttagggagggggaggggttgggccaggtgctggggaagctcccgccccgcctgccttctgcctcattctgactggctctgccctggcaaccagagcatcaggtacttacttggcagggggaggtgtctgtgggtccacccacagcaggtgtttrggactagtgcactggatggaaggagaatctcccttcccagcaccctctccacgacggtagctgtcgcccccctgtgtcgctctagcaatactgcaaggacaagttgcccatctgcttctggaccttgaaattctcttgagcctttgcagggatacagggacccaatctctgctcctcctagagcccagtacaggccaagc

SEQ ID NO: 13 - DNA, 30 bp, Artificial sequence
  Primer
acgttggatggatctgtctgaatgatgtcg

SEQ ID NO: 14 - DNA, 30 bp, Artificial sequence
  Primer
acgttggatgcagtacctcagaatctgacc

SEQ ID NO: 15 - DNA, 20 bp, Artificial sequence
  Primer
gattggatcttcacagagat

SEQ ID NO: 16 - DNA, 26 bp, Artificial sequence
  Primer
tctgaatgatgtcgttttaacttgat

SEQ ID NO: 17 - DNA, 30 bp, Artificial sequence
  Primer
acgttggatgttgttgggatagatggtggc

SEQ ID NO: 18 - DNA, 30 bp, Artificial sequence
  Probe
acgttggatgagaaaggtagatctcagtgg

SEQ ID NO: 19 - DNA, 23 bp, Artificial sequence
  Probe
agatctcagtggaatcttgcatg

SEQ ID NO: 20 - DNA, 18 bp, Artificial sequence
  Primer
aagataagaagtggtgct

SEQ ID NO: 21 - DNA, 30 bp, Artificial sequence
  Primer
acgttggatgcaggtgttcaggaataaagg

SEQ ID NO: 22 - DNA, 30 bp, Artificial sequence
  Probe
acgttggatgaagctgccaccattcgtctg

SEQ ID NO: 23 - DNA, 18 bp, Artificial sequence
  Probe
aagccttggaactcgggt

SEQ ID NO: 24 - DNA, 18 bp, Artificial sequence
  Primer
aggaagaattgtgcatgg

SEQ ID NO: 25 - DNA, 30 bp, Artificial sequence
  Primer
acgttggatgaagagaaagtggaggaagcg

SEQ ID NO: 26 - DNA, 30 bp, Artificial sequence
  Probe
acgttggatgatcacatcccatcactcctc

SEQ ID NO: 27 - DNA, 16 bp, Artificial sequence
  Probe
ctccctgtccttgaac

SEQ ID NO: 28 - DNA, 21 bp, Artificial sequence
  Primer
agcaagtgcaaaagtatggaa

SEQ ID NO: 29 - DNA, 30 bp, Artificial sequence
  Primer
acgttggatgacattccatccagcaaccac

SEQ ID NO: 30 - DNA, 30 bp, Artificial sequence
  Primer
acgttggatgcagccacgcagtgatgaaag

SEQ ID NO: 31 - DNA, 16 bp, Artificial sequence
  Primer
aggcctcgtgaagaag

SEQ ID NO: 32 - DNA, 16 bp, Artificial sequence
  Primer
accaccaaggtcattt

SEQ ID NO: 33 - DNA, 30 bp, Artificial sequence
  Primer
acgttggatgcgtggggagactaaaatgtg

SEQ ID NO: 34 - DNA, 30 bp, Artificial sequence
  Primer
acgttggatgcttactgagcccctttgaag

SEQ ID NO: 35 - DNA, 20 bp, Artificial sequence
  Primer
gcccctttgaagtgctaggc

SEQ ID NO: 36 - DNA, 17 bp, Artificial sequence
  Primer
tgattacacacagggag

SEQ ID NO: 37 - DNA, 30 bp, Artificial sequence
  Primer
acgttggatgaattcctggacattgaccgc

SEQ ID NO: 38 - DNA, 30 bp, Artificial sequence
  Primer
acgttggatgtgctaggacagcagagtaag

SEQ ID NO: 39 - DNA, 17 bp, Artificial sequence
  Primer
tccaaattaccatggga

SEQ ID NO: 40 - DNA, 22 bp, Artificial sequence
  Primer
tggacattgaccgctctcaagg

SEQ ID NO: 41 - DNA, 30 bp, Artificial sequence
  Primer
acgttggatgtctttaaattgcaccccagg

SEQ ID NO: 42 - DNA, 30 bp, Artificial sequence
  Primer
acgttggatgctctagcataatgccaagtg

SEQ ID NO: 43 - DNA, 22 bp, Artificial sequence
  Primer
agaagtttatgagatcacattc

SEQ ID NO: 44 - DNA, 19 bp, Artificial sequence
  Primer
ccccagggattataggcac

SEQ ID NO: 45 - DNA, 30 bp, Artificial sequence
  Primer
acgttggatgaatgccagctctggagtaag

SEQ ID NO: 46 - DNA, 30 bp, Artificial sequence
  Primer
acgttggatgatcaactcaggaattctctc

SEQ ID NO: 47 - DNA, 24 bp, Artificial sequence
  Primer
gaagttatttttcttttccaaatt

SEQ ID NO: 48 - DNA, 24 bp, Artificial sequence
  Primer
gagtaaggaatattgtcatcacca

SEQ ID NO: 49 - DNA, 30 bp, Artificial sequence
  Primer
acgttggatgattccctttagctgtggctg

SEQ ID NO: 50 - DNA, 30 bp, Artificial sequence
  Primer
acgttggatgcatgtgtagagagcatcgtg

SEQ ID NO: 51 - DNA, 20 bp, Artificial sequence
  Primer
gaaaatatatgggctgtgtg

SEQ ID NO: 52 - DNA, 27 bp, Artificial sequence
  Primer
tagctgtggctggccattcacctgacg

SEQ ID NO: 53 - DNA, 30 bp, Artificial sequence
  Primer
acgttggatggactaaatgtctccccttac

SEQ ID NO: 54 - DNA, 31 bp, Artificial sequence
  Primer
acgttggatgcatatatttgatgacccttcc

SEQ ID NO: 55 - DNA, 19 bp, Artificial sequence
  Primer
ccttccttacaaagcaaac

SEQ ID NO: 56 - DNA, 20 bp, Artificial sequence
  Primer
ttactgaaagaaggccaaaa

SEQ ID NO: 57 - DNA, 30 bp, Artificial sequence
  Primer
acgttggatgattctccttccatccagtgc

SEQ ID NO: 58 - DNA, 30 bp, Artificial sequence
  Primer
acgttggatgtcaggtacttacttggcagg

SEQ ID NO: 59 - DNA, 16 bp, Artificial sequence
  Primer
cccacagcaggtgttt

SEQ ID NO: 60 - DNA, 18 bp, Artificial sequence
  Primer
catccagtgcactagtcc

(19) United States
(12) Patent Application Publication
(10) Pub. No.: US 2008/0226766 A1
(43) Pub. Date: Sep. 18, 2008

(54) Dog Periodontitis

(75) Inventors: Neale Fretwell, Nottingham (GB); James Daniel Ibbotson, Nottingham (GB); Paul Glyn Jones, Nottingham (GB); Alan James Martin, Nottingham (GB)

Correspondence Address: FULBRIGHT & JAWORSKI, LLP, 1301 MCKINNEY, SUITE 5100, HOUSTON, TX, 77010-3095, US

(73) Assignee: MARS INCORPORATED, Mclean, VA (US)

(21) Appl. No.: 12/067,072

(22) PCT Filed: Sep. 18, 2006
(86) PCT No.: PCT/GB2006/003478

§ 371 Date: Apr. 29, 2008

(30) Foreign Application Priority Data
Sep. 16, 2005 (GB) 0518959.2

(51) Int. Cl. A23K 1/18; C12Q 1/68; A61K 39/02; G06F 19/00; A23L 3/34; A61P 1/00

(52) U.S. Cl. 426/2; 435/6; 424/234.1; 426/531; 426/532; 702/19

(57) ABSTRACT

A method of determining susceptibility to periodontitis in a Shih Tzu dog, Yorkshire Terrier dog or a dog of a breed that is genetically related to the Shih Tzu or Yorkshire Terrier breed, comprises: a) typing the nucleotide present in the genome of the dog at or at a position equivalent to each of the following: position 201 of SEQ ID NO: 2 (SNP_02), or a position that is in linkage disequilibrium with this position,—position 201 of SEQ ID NO: 4 (SNP_04), or a position that is in linkage disequilibrium with this position, and position 201 of SEQ ID NO: 9 (SNPJ39), or a position that is in linkage disequilibrium with this position, and b) thereby determining whether the dog is susceptible to periodontitis.

### FIELD OF THE INVENTION

The present invention relates to methods for identifying dogs susceptible to periodontitis.

### BACKGROUND OF THE INVENTION

Periodontitis may be considered a progressive form of untreated gingivitis, leading to the destruction of tissues surrounding and supporting the teeth, such as the periodontal ligament and alveolar bone. Although gingivitis is reversible, periodontitis is not, as the destruction of tooth-supporting structures results in loss of a variable level of attachment with the alveolar bone, leading to subsequent exfoliation.

The focus of research investigating the causative factors of periodontal disease has long been directed against the bacterial constituents of the plaque and their specific virulence factors that influence disease onset and progression. It has been established that the bacterial colonisation, clinically detected in the form of expanding plaque, is a dynamic process involving a number of different pathogenic genera, where the progressive dominance of anaerobic species is indicative of later disease stages.

### SUMMARY OF THE INVENTION

The present inventors have discovered single nucleotide polymorphisms (SNPs) that are associated with susceptibility to periodontitis in dogs. The identification of these polymorphisms provides the basis for diagnostic tests to identify dogs at risk of periodontitis by screening for specific molecular markers. Dogs that are determined to be susceptible to disease may then follow preventative methodologies such as appropriate diet, monitoring and maintaining good oral hygiene, in order to delay or prevent the onset of periodontitis.

Accordingly, the invention provides a method of determining susceptibility to periodontitis in a Shih Tzu dog, Yorkshire Terrier dog or a dog of a breed that is genetically related to the Shih Tzu or Yorkshire Terrier breed, the method comprising:

(a) typing the nucleotide present in the genome of the dog at or at a position equivalent to each of the following:
position 201 of SEQ ID NO: 2 (SNP_{—}02), or a position that is in linkage disequilibrium with this position,
position 201 of SEQ ID NO: 4 (SNP_{—}04), or a position that is in linkage disequilibrium with this position, and
position 201 of SEQ ID NO: 9 (SNP_{—}09) or a position that is in linkage disequilibrium with this position, and

(b) thereby determining whether the dog is susceptible to periodontitis;
a method of determining susceptibility to periodontitis in a dog, the method comprising:

(a) typing the nucleotide present in the genome of the dog at or at a position equivalent to one or more of the following:
position 201 of SEQ ID NO: 1 (SNP_{—}01), or a position that is in linkage disequilibrium with this position,
position 201 of SEQ ID NO: 2 (SNP_{—}02), or a position that is in linkage disequilibrium with this position,
position 201 of SEQ ID NO: 3 (SNP_{—}03), or a position that is in linkage disequilibrium with this position,
position 201 of SEQ ID NO: 4 (SNP_{—}04), or a position that is in linkage disequilibrium with this position,
position 201 of SEQ ID NO: 5 (SNP_{—}05), or a position that is in linkage disequilibrium with this position,
position 201 of SEQ ID NO: 6 (SNP_{—}06), or a position that is in linkage disequilibrium with this position,
position 201 of SEQ ID NO: 7 (SNP_{—}07), or a position that is in linkage disequilibrium with this position,
position 201 of SEQ ID NO: 8 (SNP_{—}08), or a position that is in linkage disequilibrium with this position,
position 201 of SEQ ID NO: 9 (SNP_{—}09), or a position that is in linkage disequilibrium with this position,
position 201 of SEQ ID NO: 10 (SNP_{—}10), or a position that is in linkage disequilibrium with this position,
position 201 of SEQ ID NO: 11 (SNP_{—}11), or a position that is in linkage disequilibrium with this position, or
position 201 of SEQ ID NO: 12 (SNP_{—}12), or a position that is in linkage disequilibrium with this position; and

(b) thereby determining whether the dog is susceptible to periodontitis;
a method of preparing customised food for a dog that is susceptible to periodontitis, the method comprising:

(a) determining whether the dog is susceptible to periodontitis by a method according to a method of the invention; and

(b) preparing food suitable for the dog;
a method of providing a customised dog food, the method comprising providing food suitable for a dog that is susceptible to periodontitis to the dog, the dog's owner or the person responsible for feeding the dog, wherein the dog has been genetically determined to be susceptible to periodontitis;
use of a compound which is therapeutic for periodontitis in the manufacture of a medicament for the prevention or treatment of periodontitis in a dog that has been identified as being susceptible to periodontitis by a method of the invention;
a method of treating a dog for periodontitis, the method comprising administering to the dog an effective amount of a therapeutic compound which prevents or treats periodontitis, wherein the dog has been identified as being susceptible to periodontitis by a method of the invention;
a method of providing care recommendations for a dog, the method comprising:

(a) determining whether the dog is susceptible to periodontitis by a method of the invention; and

(b) providing appropriate care recommendations to the dog's owner or carrier;
a database comprising information relating to polymorphisms in the canine genome and their association with periodontitis;
a method for determining susceptibility to periodontitis in a dog, the method comprising:

(a) inputting data of the nucleotide present at one or more polymorphic positions in the dog's genome to a computer system;

(b) comparing the data to a computer database, which database comprises information relating to canine genomic polymorphisms and their association with periodontitis; and

(c) determining on the basis of the comparison whether the dog is susceptible to periodontitis;
a computer program comprising program code means for performing all the steps of a method of the invention when said program is run on a computer;
a computer program product comprising program code means stored on a computer readable medium for performing a method of the invention when said program product is run on a computer;
a computer program product comprising program code means on a carrier wave, which program code means, when executed on a computer system, instruct the computer system to perform a method of the invention;
a computer system arranged to perform a method of the invention comprising:

(a) means for receiving data of the nucleotide present at one or more polymorphic positions in the dog's genome;

(b) a module for comparing the data with a database comprising information relating to canine genomic polymorphisms and their association with susceptibility to periodontitis; and

(c) means for determining on the basis of said comparison whether the dog is susceptible to periodontitis; and
a kit for carrying out the method of the invention comprising means for detecting a polymorphism associated with susceptibility to periodontitis. BRIEF DESCRIPTION OF THE SEQUENCES

SEQ ID NO: 1 shows the polynucleotide sequence encompassing SNP_{—}01.

SEQ ID NOs: 2 to 12 show the polynucleotide sequences encompassing SNP_{—}02 to SNP_{—}12, respectively.

SEQ ID NOs: 13 to 60 show the primer and primer extension sequences for genotyping SNP_{—}01 to SNP_{—}12.

### BRIEF DESCRIPTION OF THE DRAWINGS

FIG. 1 shows the age distribution of dogs sampled.

FIG. 2 shows the distribution of disease stage against number of animals sampled.

FIG. 3 shows susceptible/normal probabilities in Labradors.

FIG. 4 shows susceptible/normal probabilities in Yorkies.

FIG. 5 shows susceptible/normal probabilities in Shih Tzu.

FIG. 6 shows a rule for predicting susceptibility to periodontitis in small dog breeds.

FIG. 7 shows a simplified rule for predicting susceptibility to periodontitis in small dog breeds.

FIG. 8 illustrates schematically an embodiment of functional components arranged to carry out a method of the present invention.

FIG. 9 shows a genetic relatedness matrix for dog breeds that are genetically related to Yorkshire Terrier dogs across the 12 SNPs of the invention.

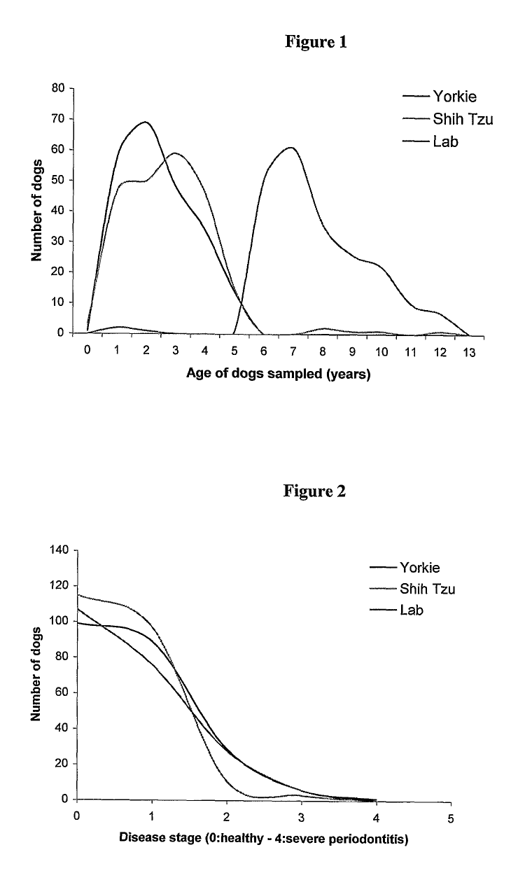

### DETAILED DESCRIPTION OF THE INVENTION

The present invention provides a method for determining whether or not a dog is susceptible to periodontitis. Susceptible to periodontitis means that there is a likelihood that a dog will develop periodontitis. More specifically, there is a likelihood that a dog will develop early onset periodontitis. The dog will therefore probably develop periodontitis at an earlier age than what would be normal for a dog of that breed.

The present inventors have found that dogs that are susceptible to periodontitis typically develop periodontitis by the age of about 5 years old, such as by the age of about 4, 3.5 or 3 years old. A particularly susceptible dog may develop periodontitis as early as before the age of about 2.5, 2 or 1.5 years old. Accordingly a dog that is susceptible to periodontitis may develop the condition at an age of from 0 to 5 years old, from 0 to 4 years old, from 0 to 3 years old, or from 0 to 2 years old.

A biological sample is taken from the dog to be tested at an early age, for example between 0 and 3 years old, between 0 and 2 years old, between 0 and 1 year old. A dog can be tested between 0 and 2 years old, more preferably between 0 and 1 year old. Accordingly, typing the SNPs of the invention can be carried out on a sample taken from the dog before the dog is about 2 years old. This gives an indication as to whether the dog will develop early onset periodontitis before the age of about 5 years old, or before the age of 4, for example between 2 and 5 or between 2 and 4 years of age. The dog can be tested at any time from 0 to 2 years old, for example 2 months old or more, 3 months old or more, or 6 months old or more. Preferably the dog is tested at as young an age as possible, for example within the first year of its life. The dog is preferably tested before any symptoms of periodontitis are apparent. The aim is therefore to take preventative measures before periodontitis occurs, such as modifying the dog's diet or improving oral hygiene, in order to delay or prevent the onset of periodontitis.

A dog of any breed may be tested by a method of the present invention. The table below provides examples of dog breeds, wherein S=small, M=medium, L=large and XL=extra large.

| | |
|---|---|
| Breed | Size |

a) Hounds

| | |
|---|---|
| Afghan Hound | L |
| Basenji | M |
| Basset Bleu De Gascogne | M |
| Basset Fauve De Bretagne | M |
| Basset Griffon Vendeen (Grand) | M |
| Basset Griffon Vendeen (Petit) | M |
| Basset Hound | M |
| Bavarian Mountain Hound | M |
| Beagle | M |
| Bloodhound | L |
| Borzoi | L |
| Dachshund | M |
| Dachshund (Long Haired) | M |
| Dachshund (Miniature Long Haired) | S |
| Dachshund (Short Haired) | M |
| Dachshund (Smooth Haired) | M |
| Dachshund (Miniature Smooth Haired) | S |
| Dachshund (Wire Haired) | M |
| Dachshund (Miniature Wire Haired) | S |
| Deerhound | L |
| Norwegian Elkhound | L |
| Finnish Spitz | M |
| Foxhound | L |
| Grand Bleu De Gascogne | L |
| Greyhound | L |
| Hamiltonstovare | L |
| Ibizan Hound | L |
| Irish Wolfhound | XL |
| Norwegian Lundehund | M |
| Otterhound | L |
| Pharaoh Hound | L |
| Rhodesian Ridgeback | L |
| Saluki | L |
| Segugio Italiano | L |
| Sloughi | L |
| Whippet | M |

b) Working Dogs

| | |
|---|---|
| Alaskan Malamute | L |
| Beauceron | L |
| Bernese Mountain Dog | XL |
| Bouvier Des Flandres | L |
| Boxer | L |
| Bullmastiff | L |
| Canadian Eskimo Dog | L |
| Dobermann | L |
| Dogue de Bordeaux | L |
| German Pinscher | M |
| Greenland Dog | L |
| Giant Schnauzer | L |
| Great Dane | XL |
| Hovawart | L |
| Leonberger | XL |
| Mastiff | XL |
| Neapolitan Mastiff | XL |
| Newfoundland | XL |
| Portuguese Water Dog | L |
| Rottweiler | L |
| Russian Black Terrier | L |
| St. Bernard | XL |
| Siberian Husky | L |
| Tibetan Mastiff | XL |

c) Terrier

| | |
|---|---|
| Airedale Terrier | L |
| Australian Terrier | S |
| Bedlington Terrier | M |
| Border Terrier | S |
| Bull Terrier | M |
| Bull Terrier (Miniature) | M |
| Cairn Terrier | S |
| Cesky Terrier | M |
| Dandie Dinmont Terrier | M |
| Fox Terrier (Smooth) | M |
| Fox Terrier (Wire) | M |
| Glen of Imaal Terrier | M |
| Irish Terrier | M |
| Jack Russell Terrier | M |
| Kerry Blue Terrier | M |
| Lakeland Terrier | M |
| Manchester Terrier | M |
| Norfolk Terrier | S |
| Norwich Terrier | S |
| Parson Russell Terrier | M |
| Scottish Terrier | M |
| Sealyham Terrier | M |
| Skye Terrier | M |
| Soft Coated Wheaten Terrier | M |
| Staffordshire Bull Terrier | M |
| Welsh Terrier | M |
| West Highland White Terrier | S |

d) Gundogs (Sporting Group)

| | |
|---|---|
| Bracco Italiano | L |
| Brittany | M |
| English Setter | L |
| German Longhaired Pointer | L |
| German Shorthaired Pointer | L |
| German Wirehaired Pointer | L |
| Gordon Setter | L |
| Hungarian Vizsla | L |
| Hungarian Wirehaired Vizsla | L |
| Irish Red and White Setter | L |
| Irish Setter | L |
| Italian Spinone | L |
| Kooikerhondje | M |
| Lagotto Romagnolo | M |
| Large Munsterlander | L |
| Nova Scotia Duck Tolling Retriever | M |
| Pointer | L |
| Retriever (Chesapeake Bay) | L |
| Retriever (Curly Coated) | L |
| Retriever (Flat Coated) | L |
| Retriever (Golden) | L |
| Retriever (Labrador) | L |
| Spaniel (American Cocker) | M |
| Spaniel (American Water) | M |
| Spaniel (Clumber) | L |
| Spaniel (Cocker) | M |
| Spaniel (English Cocker) | M |
| Spaniel (English Springer) | M |
| Spaniel (Field) | M |
| Spaniel (Irish Water) | M |
| Spaniel (Sussex) | M |
| Spaniel (Welsh Springer) | M |
| Spanish Water Dog | M |
| Vizsla | M |
| Weimaraner | L |

e) Pastoral (Herding Group)

| | |
|---|---|
| Anatolian Shepherd Dog | L |
| Australian Cattle Dog | M |
| Australian Shepherd | L |
| Bearded Collie | L |
| Belgian Shepherd Dog (Groenendael) | L |
| Belgian Shepherd Dog (Malinois) | L |
| Belgian Shepherd Dog (Laekenois) | L |
| Belgian Shepherd Dog (Tervueren) | L |
| Bergamasco | L |
| Border Collie | M |
| Briard | L |
| Collie (Rough) | L |
| Collie (Smooth) | L |
| Estrela Mountain Dog | XL |
| Finnish Lapphund | M |
| German Shepherd Dog (Alsatian) | L |
| Hungarian Kuvasz | L |
| Hungarian Puli | M |
| Komondor | L |
| Lancashire Heeler | S |
| Maremma Sheepdog | L |
| Norwegian Buhund | M |
| Old English Sheepdog | L |
| Polish Lowland Sheepdog | M |
| Pyrenean Mountain Dog | XL |
| Pyrenean Sheepdog | M |
| Samoyed | L |
| Shetland Sheepdog | M |
| Swedish Lapphund | M |
| Swedish Vallhund | M |
| Welsh Corgi (Cardigan) | M |
| Welsh Corgi (Pembroke) | M |

f) Utility Dogs (Non-sporting)

| | |
|---|---|
| Akita | L |
| American Eskimo | M |
| Boston Terrier | S |
| Bulldog | M |
| Canaan Dog | L |
| Chow Chow | L |
| Dalmatian | L |
| French Bulldog | S |
| German Spitz (Klein) | S |
| German Spitz (Mittel) | M |
| Japanese Shiba Inu | M |
| Japanese Spitz | M |
| Keeshond | M |
| Lhasa Apso | S |
| Mexican Hairless | M |
| Miniature Schnauzer | S |
| Poodle (Miniature) | M |
| Poodle (Standard) | L |
| Poodle (Toy) | S |
| Schipperke | S |
| Schnauzer (Standard) | M |
| Shar Pei | M |
| Shih Tzu | S |
| Tibetan Spaniel | S |
| Tibetan Terrier | M |

g) Toy Dogs

| | |
|---|---|
| Affenpinscher | S |
| Australian Silky Terrier | S |
| Bichon Frise | S |
| Bolognese | S |
| Cavalier King Charles Spaniel | S |
| Chihuahua (Long Coat) | S |
| Chihuahua (Smooth Coat) | S |
| Chinese Crested | S |
| Coton De Tulear | S |
| English Toy Terrier (Black and Tan) | S |
| Griffon Bruxellios | S |
| Havanese | S |
| Italian Greyhound | S |
| Japanese Chin | S |
| King Charles Spaniel | S |
| Lowchen (Little Lion Dog) | S |
| Maltese | S |
| Miniature Pinscher | S |
| Papillon | S |
| Pekingese | S |
| Pomeranian | S |
| Pug | S |
| Silky Terrier | S |
| Toy Fox Terrier | S |
| Yorkshire Terrier | S |

In a preferred embodiment of the present invention, the dog to be tested is a Shih Tzu or Yorkshire Terrier or is a breed that is genetically related to Shih Tzus or Yorkshire Terriers. In a more preferred embodiment the dog to be tested is a Yorkshire Terrier. Example 3 demonstrates how a breed that is genetically related to Yorkshire Terrier, or any other reference breed, may be determined. Specifically, the 12 SNPs of the invention are first genotyped in samples from purebred Yorkshire Terrier dogs and the allelic frequencies of each SNP are calculated. The allelic frequency of a SNP is determined by calculating the frequency of either one of the SNP alleles in approximately between 10 and 35 different purebred Yorkshire Terriers. To determine whether a breed of interest is genetically related to the Yorkshire Terrier breed across these 12 SNPs, the same 12 SNPs are genotyped in approximately between 10 and 35 purebred dogs of the breed that is being compared. The allelic frequency of each of the 12 SNPs is calculated for this breed. The degree of genetic relatedness is determined by comparing the allelic frequencies of each of the 12 SNPs between the breed of interest and the Yorkshire Terrier breed (or any other reference breed).

A metric value (measure of relatedness) is calculated for each SNP by finding the difference in allele frequency between that in the breed of interest and that in the reference breed. The squares of these values are then summed for each SNP. This provides a metric value of relatedness for the two breeds. A metric value that demonstrates sufficient genetic relatedness between breeds may be a value of 0.4 or less, for example 0.3 or less, 0.2 or less, or 0.1 or less.

A genetic matrix can be constructed for multiple different breeds to determine how genetically related they are to the Yorkshire Terrier or Shih Tzu breed, by repeating the comparison of SNP allele frequencies between breeds as described above for every possible combination of breeds of interest.

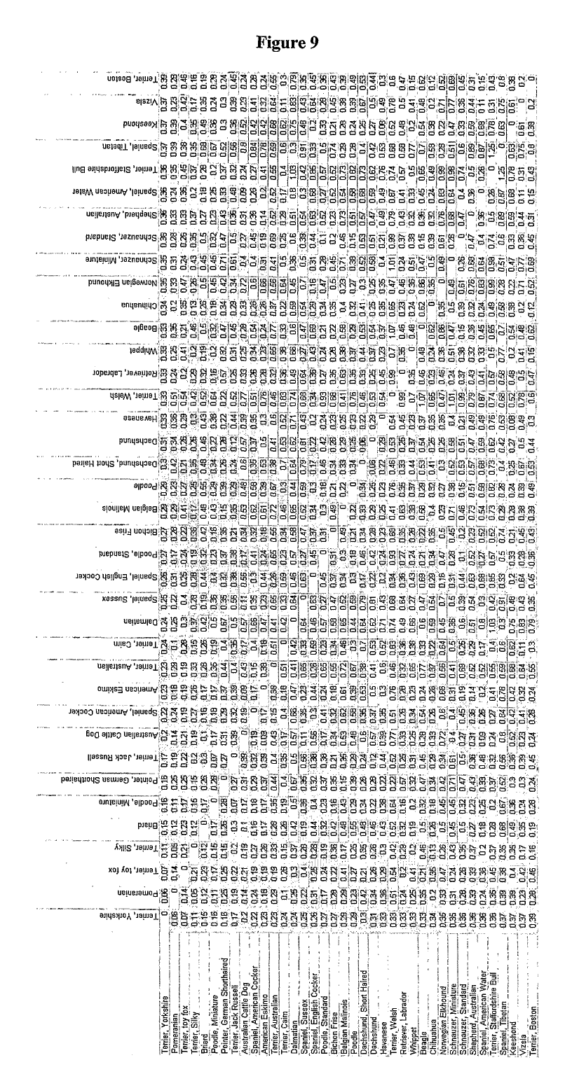

An example of a matrix produced using the 12 SNPs of the invention and a variety of breeds is shown in FIG. 9. In this Figure, all of the breeds are genetically related to Yorkshire Terrier because they have metric values of less than 0.4 when compared with the Yorkshire Terrier breed.

Accordingly, in an embodiment of the present invention a breed that is genetically related to Yorkshire Terrier is a toy dog and is selected from Pomeranian, Toy Fox Terrier, Silky Terrier, Bichon Frise, Havanese and Chihuahua. Preferably the breed is Pomeranian, Toy Fox Terrier, Silky Terrier or Bichon Frise. More preferably the breed is Pomeranian, Toy Fox Terrier or Silky Terrier.

In another embodiment, a breed that is genetically related to Yorkshire Terrier is a terrier and is selected from Jack Russell Terrier, Australian Terrier, Cairn Terrier, Welsh Terrier and Staffordshire Bull Terrier. Preferably the breed is Jack Russell Terrier, Australian Terrier or Cairn Terrier. More preferably the breed is Jack Russell Terrier or Australian Terrier.

In another embodiment, a breed that is genetically related to Yorkshire Terrier is a pastoral dog (herding group) and is selected from Briard, Australian Cattle Dog, Belgian Malinois and Australian Shepherd. Preferably the breed is Briard, Australian Cattle Dog or Belgian Malinois. More preferably the breed is Briard or Australian Cattle Dog.

In a further embodiment, a breed that is genetically related to Yorkshire Terrier is a utility dog (non-sporting) and is selected from Miniature Poodle, American Eskimo, Dalmatian, Standard Poodle, Toy Poodle, Miniature Schnauzer, Standard Schnauzer, Tibetan Spaniel, Keeshond and Boston Terrier. Preferably the breed is Miniature Poodle, American Eskimo, Dalmatian, Standard Poodle or Toy Poodle. More preferably the breed is Miniature Poodle, American Eskimo or Dalmatian.

In another embodiment, a breed that is genetically related to Yorkshire Terrier is a gundog (sporting) and is selected from German Shorthaired Pointer, American Cocker Spaniel, Sussex Spaniel, English Cocker Spaniel, Labrador Retriever, American Water Spaniel and Viszla. Preferably the breed is German Shorthaired Pointer, American Cocker Spaniel, Sussex Spaniel, English Cocker Spaniel or Labrador Retriever. More preferably the breed is German Shorthaired Pointer, American Cocker Spaniel or Sussex Spaniel.

In another embodiment, a breed that is genetically related to Yorkshire Terrier is a hound and is selected from Short Haired Dachshund, Dachshund, Whippet, Beagle and Norwegian Elkhound. Preferably the breed is Short Haired Dachshund, Dachshund, Whippet or Beagle. More preferably the breed is Short Haired Dachshund or Dachshund.

Preferably the dog to be tested is a purebred. However, the dog may be a mixed or crossbred, or a mongrel or out-bred dog. The dog to be tested can have at least 50% Shih Tzu, Yorkshire Terrier, or a dog breed that is genetically related to the Shih Tzu or Yorkshire Terrier breed, in its genetic breed background. For example, the genetic breed background can be at least 75% Shih Tzu, Yorkshire Terrier, or breed of a dog that is genetically related to Shih Tzu or Yorkshire Terrier breed. The genetic breed background of a dog may be determined by detecting the presence or absence of two or more breed-specific SNP markers in the dog.

The present inventors have determined that it is possible to predict whether or not a dog is susceptible to periodontitis by typing one or more of the following polymorphic SNP positions: SNP_{—}01, SNP_{—}02, SNP_{—}03, SNP_{—}04, SNP_{—}05, SNP_{—}06, SNP_{—}07, SNP_{—}08, SNP_{—}09, SNP 0, SNP_{—}11 and SNP_{—}12 as defined herein.

The present invention provides a method of determining susceptibility to periodontitis in a dog, the method comprising:

a) typing the nucleotide present in the genome of the dog at or at a position equivalent to each of the following:
position 201 of SEQ ID NO: 2 (SNP_{—}02), or a position that is in linkage disequilibrium with this position,
position 201 of SEQ ID NO: 4 (SNP_{—}04), or a position that is in linkage disequilibrium with this position, and
position 201 of SEQ ID NO: 9 (SNP_{—}09) or a position that is in linkage disequilibrium with this position, and

b) thereby determining whether the dog is susceptible to periodontitis.

When 3 SNP positions are typed, this may serve as a preliminary screen for susceptibility to periodontitis. A more accurate screen involving more SNP loci, for example 4, 5, 6, 7, 8, 9, 10, 11 or 12 SNP loci can then be applied to dogs that are candidates for being susceptible to early onset periodontitis.

The inventors have further discovered models or rules for using the SNPs of the invention to determine whether or not a dog is susceptible to periodontitis that involve applying weightings to the SNP genotypes and thereby determining a susceptibility factor. It will be appreciated that many different models or rules are possible using any number or combination of the 12 SNPs, and using different weightings and constants in the formulae.

In an embodiment of the present invention, a dog is determined to be susceptible if, when a susceptibility factor is determined using the rule

X=SNP_{—}02−1.109−SNP_{—}09−SNP_{—}04

wherein:
for SNP_{—}02, AA=−1, AC=0 and CC=1,
for SNP_{—}09, AA=−1, AC=0 and CC=1, and
for SNP_{—}04, GG=−1, GA=0 and AA=1;

X is greater than or equals −2.109.

The invention may further comprise typing the nucleotide present in the genome of the dog at or at a position equivalent to each of the following:
position 201 in SEQ ID NO: 1 (SNP_{—}01) or a position that is in linkage disequilibrium with this position,
position 201 in SEQ ID NO:3 (SNP_{—}03) or a position that is in linkage disequilibrium with this position,
position 201 in SEQ ID NO:5 (SNP_{—}05) or a position that is in linkage disequilibrium with this position,
position 201 in SEQ ID NO:6 (SNP_{—}06), or a position that is in linkage disequilibrium with this position,
position 201 in SEQ ID NO:7 (SNP_{—}07) or a position that is in linkage disequilibrium with this position,
position 201 in SEQ ID NO:8 (SNP_{—}08) or a position that is in linkage disequilibrium with this position,
position 201 in SEQ ID NO:10 (SNP_{—}0) or a position that is in linkage disequilibrium with this position,
position 201 in SEQ ID NO: 11 (SNP_{—}11) or a position that is in linkage disequilibrium with this position, and
position 201 in SEQ ID NO:12 (SNP_{—}12) or a position that is in linkage disequilibrium with this position.

When all 12 SNP positions are typed, a dog is determined to be susceptible if, when a susceptibility factor is determined using the rule

X=SNP_{—}01+2*SNP_{—}02−SNP_{—}03−SNP_{—}04−SNP_{—}05−SNP_{—}06−SNP_{—}07+SNP_{—}08−SNP_{—}09−SNP_{—}10−SNP_{—}11+SNP_{—}12−0.177

wherein:
for SNP_{—}01, AA=−1, AG=0 and GG=1;
for SNP_{—}02, AA=−1, AC=0 and CC=1;
for SNP_{—}03, CC=−1, CG=0 and GG=1;
for SNP_{—}04, GG=−1, GA=0 and AA=1;
for SNP_{—}05, AA=−1, AG=0 and GG=1;
for SNP_{—}06, AA=−1, AG=0 and GG=1;
for SNP_{—}07, CC=−1, CG=0 and GG=1;
for SNP_{—}08, AA=−1, AG=0 and GG=1;
for SNP_{—}09, AA=−1, AC=0 and CC=1;
for SNP_{—}10, AA=−1, AG=0 and GG=1;
for SNP_{—}11, GG=−1, GA=0 and AA=1;
for SNP_{—}12, AA=−1, AG=0 and GG=1;

X is greater than or equals −4.177.

Any number and any combination of the 12 SNP positions as described herein may be typed to carry out the invention. Preferably at least 3 SNP positions are typed, more preferably at least 4, 5, 6, 7, 8, 9, 10, 11 or 12 positions are typed.

The polymorphic position may be typed directly, in other words by determining the nucleotide present at that position, or indirectly, for example by determining the nucleotide present at another polymorphic position that is in linkage disequilibrium with said polymorphic position.

Polymorphisms which are in linkage disequilibrium with each other in a population are typically found together on the same chromosome. Typically one is found at least 30% of the times, for example at least 40%, at least 50%, at least 70% or at least 90%, of the time the other is found on a particular chromosome in individuals in the population. Thus a polymorphism which is not a functional susceptibility polymorphism, but is in linkage disequilibrium with a functional polymorphism, may act as a marker indicating the presence of the functional polymorphism.

Polymorphisms which are in linkage disequilibrium with the polymorphisms mentioned herein are typically located within 500 kb, preferably within 400 kb, within 200 kb, within 100 kb, within 50 kb, within 10 kb, within 5 kb, within 1 kb, within 500 bp, within 100 bp, within 50 bp or within 10 bp of the polymorphism.

It will be understood that the exact sequences presented in SEQ ID NOs: 1 to 12 will not necessarily be present in the dog to be tested. The sequence and thus the position of the SNP could for example vary because of deletions or additions of nucleotides in the genome of the dog. Those skilled in the art will be able to determine a position that corresponds to position 201 in each of SEQ ID NOs: 1 to 12, using for example a computer program such as PILEUP or BLAST as referred to below.

### Detection of Polymorphisms

The detection of polymorphisms according to the invention may comprise contacting a polynucleotide or protein of the dog with a specific binding agent for a polymorphism and determining whether the agent binds to the polynucleotide or protein, wherein binding of the agent indicates the presence of the polymorphism, and lack of binding of the agent indicates the absence of the polymorphism.

The method is generally carried out in vitro on a sample obtained from the dog, where the sample contains DNA from the dog. The sample typically comprises a body fluid and/or cells of the dog and may, for example, be obtained using a swab, such as a mouth swab. The sample may be a blood, urine, saliva, skin, cheek cell or hair root sample. The sample is typically processed before the method is carried out, for example DNA extraction may be carried out. The polynucleotide or protein in the sample may be cleaved either physically or chemically, for example using a suitable enzyme. In one embodiment the part of polynucleotide in the sample is copied or amplified, for example by cloning or using a PCR based method prior to detecting the polymorphism.

In the present invention, any one or more methods may comprise determining the presence or absence of one or more polymorphisms in the dog. The polymorphism is typically detected by directly determining the presence of the polymorphic sequence in a polynucleotide or protein of the dog. Such a polynucleotide is typically genomic DNA, mRNA or cDNA. The polymorphism may be detected by any suitable method such as those mentioned below.

A specific binding agent is an agent that binds with preferential or high affinity to the protein or polypeptide having the polymorphism but does not bind or binds with only low affinity to other polypeptides or proteins. The specific binding agent may be a probe or primer. The probe may be a protein (such as an antibody) or an oligonucleotide. The probe may be labelled or may be capable of being labelled indirectly. The binding of the probe to the polynucleotide or protein may be used to immobilise either the probe or the polynucleotide or protein.

Generally in the method, a polymorphism can be detected by determining the binding of the agent to the polymorphic polynucleotide or protein of the dog. However in one embodiment the agent is also able to bind the corresponding wild-type sequence, for example by binding the nucleotides or amino acids which flank the variant position, although the manner of binding to the wild-type sequence will be detectably different to the binding of a polynucleotide or protein containing the polymorphism.

The method may be based on an oligonucleotide ligation assay in which two oligonucleotide probes are used. These probes bind to adjacent areas on the polynucleotide that contains the polymorphism, allowing after binding the two probes to be ligated together by an appropriate ligase enzyme. However the presence of single mismatch within one of the probes may disrupt binding and ligation. Thus ligated probes will only occur with a polynucleotide that contains the polymorphism, and therefore the detection of the ligated product may be used to determine the presence of the polymorphism.

In one embodiment the probe is used in a heteroduplex analysis based system. In such a system when the probe is bound to polynucleotide sequence containing the polymorphism it forms a heteroduplex at the site where the polymorphism occurs and hence does not form a double strand structure. Such a heteroduplex structure can be detected by the use of a single or double strand specific enzyme. Typically the probe is an RNA probe, the heteroduplex region is cleaved using RNAase H and the polymorphism is detected by detecting the cleavage products.

The method may be based on fluorescent chemical cleavage mismatch analysis which is described for example in PCR Methods and Applications 3, 268-71 (1994) and Proc. Natl. Acad. Sci. 85, 4397-4401 (1998).

In one embodiment a PCR primer is used that primes a PCR reaction only if it binds a polynucleotide containing the polymorphism, for example a sequence-specific PCR system, and the presence of the polymorphism may be determined by detecting the PCR product. Preferably the region of the primer that is complementary to the polymorphism is at or near the 3′ end of the primer. The presence of the polymorphism may be determined using a fluorescent dye and quenching agent-based PCR assay such as the Taqman PCR detection system.

The specific binding agent may be capable of specifically binding the amino acid sequence encoded by a polymorphic sequence. For example, the agent may be an antibody or antibody fragment. The detection method may be based on an ELISA system. The method may be an RFLP based system. This can be used if the presence of the polymorphism in the polynucleotide creates or destroys a restriction site that is recognised by a restriction enzyme.

The presence of the polymorphism may be determined based on the change that the presence of the polymorphism makes to the mobility of the polynucleotide or protein during gel electrophoresis. In the case of a polynucleotide, single-stranded conformation polymorphism (SSCP) or denaturing gradient gel electrophoresis (DDGE) analysis may be used. In another method of detecting the polymorphism a polynucleotide comprising the polymorphic region is sequenced across the region that contains the polymorphism to determine the presence of the polymorphism.

The presence of the polymorphism may be detected by means of fluorescence resonance energy transfer (FRET). In particular, the polymorphism may be detected by means of a dual hybridisation probe system. This method involves the use of two oligonucleotide probes that are located close to each other and that are complementary to an internal segment of a target polynucleotide of interest, where each of the two probes is labelled with a fluorophore. Any suitable fluorescent label or dye may be used as the fluorophore, such that the emission wavelength of the fluorophore on one probe (the donor) overlaps the excitation wavelength of the fluorophore on the second probe (the acceptor). A typical donor fluorophore is fluorescein (FAM), and typical acceptor fluorophores include Texas red, rhodamine, LC-640, LC-705 and cyanine 5 (CyS).

In order for fluorescence resonance energy transfer to take place, the two fluorophores need to come into close proximity on hybridisation of both probes to the target. When the donor fluorophore is excited with an appropriate wavelength of light, the emission spectrum energy is transferred to the fluorophore on the acceptor probe resulting in its fluorescence. Therefore, detection of this wavelength of light, during excitation at the wavelength appropriate for the donor fluorophore, indicates hybridisation and close association of the fluorophores on the two probes. Each probe may be labelled with a fluorophore at one end such that the probe located upstream (5′) is labelled at its 3′ end, and the probe located downstream (3′) is labelled at is 5′ end. The gap between the two probes when bound to the target sequence may be from 1 to 20 nucleotides, preferably from 1 to 17 nucleotides, more preferably from 1 to 10 nucleotides, such as a gap of 1, 2, 4, 6, 8 or 10 nucleotides.

The first of the two probes may be designed to bind to a conserved sequence of the gene adjacent to a polymorphism and the second probe may be designed to bind to a region including one or more polymorphisms. Polymorphisms within the sequence of the gene targeted by the second probe can be detected by measuring the change in melting temperature caused by the resulting base mismatches. The extent of the change in the melting temperature will be dependent on the number and base types involved in the nucleotide polymorphisms.

### Polynucleotides

Polynucleotides of the invention may be used as a probe or primer, which is capable of selectively binding to a polymorphism. The invention thus provides a probe or primer for use in a method according to the invention, which probe or primer is capable of selectively detecting the presence of a polymorphism associated with susceptibility to periodontitis. Preferably the probe is isolated or recombinant nucleic acid. The probe may be immobilised on an array, such as a polynucleotide array.

Such primers, probes and other fragments will preferably be at least 10, preferably at least 15 or at least 20, for example at least 25, at least 30 or at least 40 nucleotides in length. They will typically be up to 40, 50, 60, 70, 100 or 150 nucleotides in length. Probes and fragments can be longer than 150 nucleotides in length, for example up to 200, 300, 400, 500, 600, 700 nucleotides in length, or even up to a few nucleotides, such as five or ten nucleotides, short of a full length polynucleotide sequence of the invention.

### Homologues

Homologues of polynucleotide or protein sequences are referred to herein. Such homologues typically have at least 70% homology, preferably at least 80, 90%, 95%, 97% or 99% homology, for example over a region of at least 15, 20, 30, 100 more contiguous nucleotides or amino acids. The homology may be calculated on the basis of nucleotide or amino acid identity (sometimes referred to as “hard homology”).

For example the UWGCG Package provides the BESTFIT program that can be used to calculate homology (for example used on its default settings) (Devereux et al (1984) Nucleic Acids Research 12, p 387-395). The PILEUP and BLAST algorithms can be used to calculate homology or line up sequences (such as identifying equivalent or corresponding sequences (typically on their default settings), for example as described in Altschul S. F. (1993) J Mol Evol 36:290-300; Altschul, S, F et al (1990) J Mol Biol 215:403-10.

Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence that either match or satisfy some positive-valued threshold score T when aligned with a word of the same length in a database sequence. T is referred to as the neighbourhood word score threshold (Altschul et al, supra). These initial neighbourhood word hits act as seeds for initiating searches to find HSPs containing them. The word hits are extended in both directions along each sequence for as far as the cumulative alignment score can be increased. Extensions for the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T and X determine the sensitivity and speed of the alignment. The BLAST program uses as default a word length (W) of 11, the BLOSUM62 scoring matrix (see Henikoff and Henikoff (1992) Proc. Natl. Acad. Sci. USA 89: 10915-10919) alignments (B) of 50, expectation (E) of 10, M=5, N=4, and a comparison of both strands.

The BLAST algorithm performs a statistical analysis of the similarity between two sequences; see e.g., Karlin and Altschul (1993) Proc. Natl. Acad. Sci. USA 90: 5873-5787. One measure of similarity provided by the BLAST algorithm is the smallest sum probability (P(N)), which provides an indication of the probability by which a match between two polynucleotide or amino acid sequences would occur by chance. For example, a sequence is considered similar to another sequence if the smallest sum probability in comparison of the first sequence to the second sequence is less than about 1, preferably less than about 0.1, more preferably less than about 0.01, and most preferably less than about 0.001.

The homologous sequence typically differs by at least 1, 2, 5, 10, 20 or more mutations, which may be substitutions, deletions or insertions of nucleotide or amino acids. These mutations may be measured across any of the regions mentioned above in relation to calculating homology. In the case of proteins the substitutions are preferably conservative substitutions. These are defined according to the following Table. Amino acids in the same block in the second column and preferably in the same line in the third column may be substituted for each other:

| | | |
|---|---|---|
| ALIPHATIC | Non-polar | G A P |
| | | I L V |
| | Polar - uncharged | C S T M |
| | | N Q |
| | Polar - charged | D E |
| | | K R |
| AROMATIC | | H F W Y |

Shorter polypeptide sequences are also within the scope of the invention. For example, a fragment of a polypeptide sequence of the invention is typically at least 10, 15, 20, 30, 40, 50, 60, 70, 80, 100, 150 or 200 amino acids in length. Polypeptides of the invention may be chemically modified, for example post-translationally modified. The polypeptides may be glycosylated or comprise modified amino acid residues. Such modified polypeptides fall within the scope of the term “polypeptide” of the invention.

The polypeptides, polynucleotides, vectors, cells or antibodies of the invention may be present in an isolated or substantially purified form. They may be mixed with carriers or diluents that will not interfere with their intended use and still be regarded as substantially isolated. They may also be in a substantially purified form, in which case they will generally comprise at least 90%, e.g. at least 95%, 98% or 99%, of the proteins, polynucleotides, cells or dry mass of the preparation.

It is understood that any of the above features that relate to polynucleotides and proteins may also be a feature of the other polypeptides and proteins mentioned herein, such as the polypeptides and proteins used in the screening and therapeutic aspects of the invention. In particular such features may be any of the lengths, modifications and vector forms mentioned above.

### Detector Antibodies

A detector antibody is an antibody that is specific for one polymorphism but does not bind to any other polymorphism as described herein. Detector antibodies are for example useful in purification, isolation or screening methods involving immunoprecipitation techniques.

Antibodies may be raised against specific epitopes of the polypeptides of the invention. An antibody, or other compound, “specifically binds” to a polypeptide when it binds with preferential or high affinity to the protein for which it is specific but does substantially bind not bind or binds with only low affinity to other polypeptides. A variety of protocols for competitive binding or immunoradiometric assays to determine the specific binding capability of an antibody are well known in the art (see for example Maddox et al, J. Exp. Med. 158, 1211-1226, 1993). Such immunoassays typically involve the formation of complexes between the specific protein and its antibody and the measurement of complex formation.

For the purposes of this invention, the term “antibody”, unless specified to the contrary, includes fragments that bind a polypeptide of the invention. Such fragments include Fv, F(ab′) and F(ab′)₂ fragments, as well as single chain antibodies. Furthermore, the antibodies and fragment thereof may be chimeric antibodies, CDR-grafted antibodies or humanised antibodies.

Antibodies may be used in a method for detecting polypeptides of the invention in a biological sample (such as any such sample mentioned herein), which method comprises:

I providing an antibody of the invention;
II incubating a biological sample with said antibody under conditions which allow for the formation of an antibody-antigen complex; and
III determining whether antibody-antigen complex comprising said antibody is formed.

Antibodies of the invention can be produced by any suitable method. Means for preparing and characterising antibodies are well known in the art, see for example Harlow and Lane (1988) “Antibodies: A Laboratory Manual”, Cold Spring Harbor Laboratory Press, Cold Spring Harbor, N.Y. For example, an antibody may be produced by raising an antibody in a host animal against the whole polypeptide or a fragment thereof, for example an antigenic epitope thereof, hereinafter the “immunogen”. The fragment may be any of the fragments mentioned herein (typically at least 10 or at least 15 amino acids long).

A method for producing a polyclonal antibody comprises immunising a suitable host animal, for example an experimental animal, with the immunogen and isolating immunoglobulins from the animal's serum. The animal may therefore be inoculated with the immunogen, blood subsequently removed from the animal and the IgG fraction purified. A method for producing a monoclonal antibody comprises immortalising cells which produce the desired antibody. Hybridoma cells may be produced by fusing spleen cells from an inoculated experimental animal with tumour cells (Kohler and Milstein (1975) Nature 256, 495-497).

An immortalized cell producing the desired antibody may be selected by a conventional procedure. The hybridomas may be grown in culture or injected intraperitoneally for formation of ascites fluid or into the blood stream of an allogenic host or immunocompromised host. Human antibody may be prepared by in vitro immunisation of human lymphocytes, followed by transformation of the lymphocytes with Epstein-Barr virus.

For the production of both monoclonal and polyclonal antibodies, the experimental animal is suitably a goat, rabbit, rat, mouse, guinea pig, chicken, sheep or horse. If desired, the immunogen may be administered as a conjugate in which the immunogen is coupled, for example via a side chain of one of the amino acid residues, to a suitable carrier. The carrier molecule is typically a physiologically acceptable carrier. The antibody obtained may be isolated and, if desired, purified.

### Detection Kit

The invention also provides a kit that comprises means for typing one or more polymorphisms in a dog that are associated with susceptibility to periodontitis. In particular, such means may include a specific binding agent, probe, primer, pair or combination of primers, or antibody, including an antibody fragment, as defined herein which is capable of detecting or aiding detection of a polymorphism. The primer or pair or combination of primers may be sequence specific primers that only cause PCR amplification of a polynucleotide sequence comprising the polymorphism to be detected, as discussed herein. The kit may also comprise a specific binding agent, probe, primer, pair or combination of primers, or antibody that is capable of detecting the absence of the polymorphism. The kit may further comprise buffers or aqueous solutions.

The kit may additionally comprise one or more other reagents or instruments that enable any of the embodiments of the method mentioned above to be carried out. Such reagents or instruments may include one or more of the following: a means to detect the binding of the agent to the polymorphism, a detectable label such as a fluorescent label, an enzyme able to act on a polynucleotide, typically a polymerase, restriction enzyme, ligase, RNAse H or an enzyme which can attach a label to a polynucleotide, suitable buffer(s) or aqueous solutions for enzyme reagents, PCR primers which bind to regions flanking the polymorphism as discussed herein, a positive and/or negative control, a gel electrophoresis apparatus, a means to isolate DNA from sample, a means to obtain a sample from the individual, such as swab or an instrument comprising a needle, or a support comprising wells on which detection reactions can be carried out. The kit may be, or include, an array such as a polynucleotide array comprising the specific binding agent, preferably a probe, of the invention. The kit typically includes a set of instructions for using the kit.

### Treatment of Periodontitis

The invention provides a method of treating dog for periodontitis, the method comprising identifying a dog which is susceptible to periodontitis by a method of the invention, and administering to the dog an effective amount of a therapeutic agent which treats periodontitis. The therapeutic agent may be an antibacterial, such as chlorhexidine or a sulfadiazine, which may optionally be formulated as a spray or gel. The agent may be a root irrigant, such as hypochlorite, iodine or fluoride, or a dental paint, which may be topically applied to reduce plaque formation. Other suitable therapeutic agents include antibiotics, for example monocycline, which may optionally be applied locally; anti-cyclooxygenase-2 (COX-2) therapy, for example meloxicam; systemic or local non-steroidal anti-inflammatories (NSAIDS), for example indomethicin; corticosteroids, which may optionally be applied locally; hyaluronan, which may be formulated as a topical gel; Periostat-systemic doxycycline, which is typically administered at a sub-antimicrobial dose; bisphosphonates, for example disodium chlodronate; nitric oxide synthase inhibitors (iNOS), for example aminoguanidine; vaccination against P. gingivalis; or any drug known in the art that may be used to treat periodontitis.

The therapeutic agent may be administered in various manners such as orally, intracranially, intravenously, intramuscularly, intraperitoneally, intranasally, intradermally, and subcutaneously. The pharmaceutical compositions that contain the therapeutic agent will normally be formulated with an appropriate pharmaceutically acceptable carrier or diluent depending upon the particular mode of administration being used. For instance, parenteral formulations are usually injectable fluids that use pharmaceutically and physiologically acceptable fluids such as physiological saline, balanced salt solutions, or the like as a vehicle. Oral formulations, on the other hand, may be solids, for example tablets or capsules, or liquid solutions or suspensions. In a preferred embodiment, the therapeutic agent is administered to the dog in its diet, for example in its drinking water or food.

The amount of therapeutic agent that is given to a dog will depend upon a variety of factors including the age of the dog under treatment and the severity of the condition. A typical daily dose is from about 0.1 to 50 mg per kg, preferably from about 0.1 mg/kg to 10 mg/kg of body weight, according to the activity of the drug, the age, weight and condition of the dog to be treated, the severity of the disease and the frequency and route of administration. Preferably, daily dosage levels are from 5 mg to 2 g.

### Customised Food

In one aspect, the invention relates to a customised diet for a dog that is susceptible to periodontitis. Such a food may be in the form of, for example, wet pet foods, semi-moist pet foods, dry pet foods and pet treats. Wet pet food generally has a moisture content above 65%. Semi-moist pet food typically has a moisture content between 20-65% and can include humectants and other ingredients to prevent microbial growth. Dry pet food, also called kibble, generally has a moisture content below 20% and its processing typically includes extruding, drying and/or baking in heat. The ingredients of a dry pet food generally include cereal, grains, meats, poultry, fats, vitamins and minerals. The ingredients are typically mixed and put through an extruder/cooker. The product is then typically shaped and dried, and after drying, flavours and fats may be coated or sprayed onto the dry product.

Accordingly, the present invention enables the preparation of customised food suitable for a dog that is susceptible to periodontitis, wherein the customised food formulation comprises ingredients that prevent or alleviate periodontitis, and/or does not comprise components that contribute to or aggravate periodontitis. Such ingredients may be any of those known in the art to prevent or alleviate periodontitis. The preparation of customised dog food may be carried out by electronic means, for example by using a computer system.

In one embodiment, the customised food may be formulated to include functional or active ingredients that may prevent or alleviate periodontitis. Such an ingredient may be a compound that stimulates the immune response, relieves inflammation or that has an antimicrobial action. Examples of such active or functional ingredients include antimicrobial natural oils, such as eucalyptus, tea tree, rosemary and thyme; zinc, which may act to inhibit microbial metabolism; polyphosphates (STPP), which may increase calcium sequestration to prevent tartar; furanones (from seaweed); green tea, which comprises anti-inflammatory polyphenolics; borage oil, which comprises omega 3 fatty acids; anti-oxidants such as vitamin E; aloe vera, which may be anti-inflammatory and promote healing; co-enzyme Q10, which has been observed to be present at lower levels in human periodontitis, and may promote healing; vitamin C, which may promote collagen formation, and low levels of which have been linked to increased risk of periodontitis; and folic acid, which may help to promote gingival health.

The present invention also relates to a method of providing a customised dog food, comprising providing food suitable for a dog which is susceptible to periodontitis to the dog, the dog's owner or the person responsible for feeding the dog, wherein the dog has been determined to be susceptible to periodontitis by a method of the invention. In one aspect of the invention, the customised food is made to inventory and supplied from inventory, i.e. the customised food is pre-manufactured rather than being made to order. Therefore according to this aspect of the invention the customised food is not specifically designed for one particular dog but instead is suitable for more than one dog. For example, the customised food may be suitable for any dog that is susceptible to periodontitis. Alternatively, the customised food may be suitable for a sub-group of dogs that are susceptible to periodontitis, such as dogs of a particular breed, size or lifestage. In another embodiment, the food may be customised to meet the nutritional requirements of an individual dog.

The present invention further relates to pet care products that are suitable for dogs that are susceptible to periodontitis, or which help to prevent or alleviate the symptoms or causes of periodontitis. For example, such products may include pet treats that promote good oral hygiene, such as oral care chews or rawhide chews, or other oral hygiene products such as a toothbrush or toothpaste. A toothpaste suitable for a dog susceptible to periodontitis may contain an antimicrobial agent, for example triclosan. The invention also relates to a method of providing care recommendations to a dog's owner or carrier, for example to carry out a scale and root plane every 6 months to one year.

### Bioinformatics

The sequences of the polymorphisms may be stored in an electronic format, for example in a computer database. Accordingly, the invention provides a database comprising information relating to polymorphic sequences associated with susceptibility to periodontitis in dogs. The database may include further information about the polymorphism, for example the degree of association of the polymorphism with susceptibility to periodontitis.

A database as described herein may be used to determine the susceptibility of a dog to periodontitis. Such a determination may be carried out by electronic means, for example by using a computer system (such as a PC). Typically, the determination will be carried out by inputting genetic data from the dog to a computer system; comparing the genetic data to a database comprising information relating to polymorphism associated with disease susceptibility; and on the basis of this comparison, determining the susceptibility of the dog to periodontitis.

The invention also provides a computer program comprising program code means for performing all the steps of a method of the invention when said program is run on a computer. Also provided is a computer program product comprising program code means stored on a computer readable medium for performing a method of the invention when said program is run on a computer. A computer program product comprising program code means on a carrier wave that, when executed on a computer system, instruct the computer system to perform a method of the invention is additionally provided.

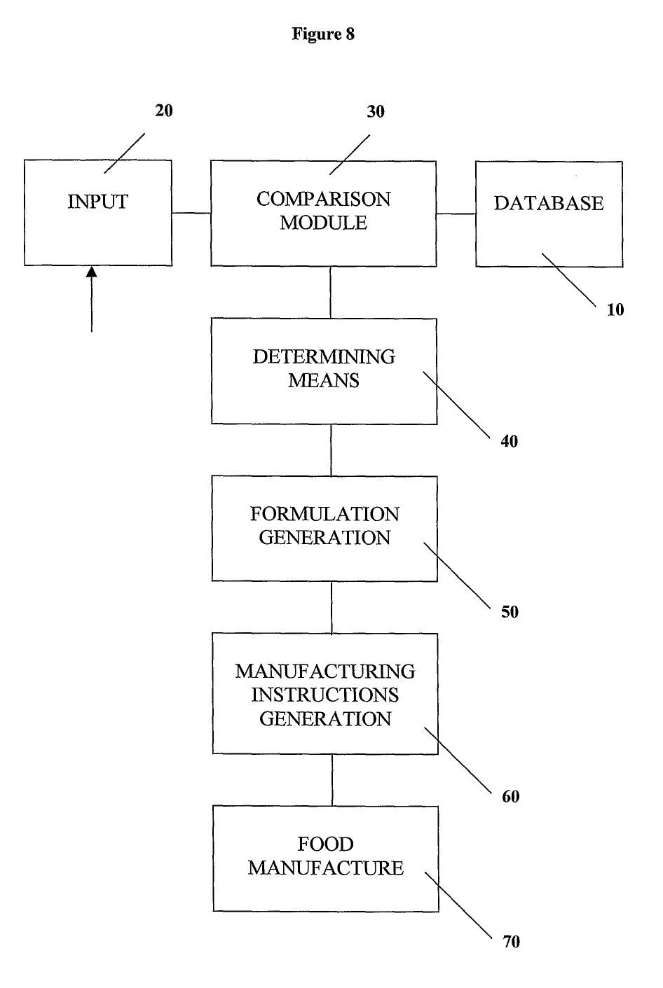

As illustrated in FIG. 8, the invention also provides an apparatus arranged to perform a method according to the invention. The apparatus typically comprises a computer system, such as a PC. In one embodiment, the computer system comprises: means 20 for receiving genetic data from the dog; a module 30 for comparing the data with a database 10 comprising information relating to polymorphisms; and means 40 for determining on the basis of said comparison whether or not the dog is susceptible to periodontitis.

The invention is illustrated by the following Examples:

### EXAMPLE 1

### Materials and Methods

### Sample Collection

The entire historical computerised PetWare database of Banfield, The Pet Hospital network was searched using owner geography (Virginia, Maryland, Pennsylvania, Florida or Georgia), breed and age as search strings to identify suitable client owned pets as candidates for the study. A telephone interview was conducted with each owner to confirm that the dog was eligible for the study based upon the inclusion and exclusion criteria defined below. Study-eligible dogs were scheduled to visit their local Banfield clinic and a conscious oral and physical examination was conducted using the protocol described. A dedicated trained dental technician performed all phenotypic assessments undertaken to ensure objectivity of the conscious scoring scheme used.

### Study Recruitment Inclusion and Exclusion Criteria

Dogs were excluded from the study if they failed to qualify for the study using the following criteria (verified on presentation for Veterinary assessment at the clinic):
Breed of dog—only Labrador Retrievers, Shih Tzus, Yorkshire Terriers and Poodles (toy, miniature or standard) were included
Age of dog—Labradors from 6 to 12 years old; Yorkshire Terriers, Poodles and Shih Tzus aged from 1 to 6 years old
No regular oral prophylaxis or oral prophylaxis within the last 3 months
No regular medication or treatment with chronic steroids
No renal failure, endocrine disorders or other systemic disease
No treatment for diabetes

A clearly defined protocol was followed for the oral assessment and scoring procedure. A small blood sample was taken by venopuncture for clinical blood chemistry and haematology analysis. A health check was conducted by the resident Veterinarian to monitor the potential presence of additional concurrent disease. Blood samples were drawn into 6 ml EDTA-coated blood tubes, which were stored at −20° C. until ready for subsequent genomic DNA extraction.

### Phenotypic Assessment

Phenotypic assessment was performed for each dog enrolled in the study by the same trained technician to give objectivity to the scoring criteria, using an overall conscious assessment of the periodontal status of the dog as detailed below:
Stage 0—no evidence disease present, healthy gums are pink in colour and have crisp even margins
Stage 1—the margin of the attached gingiva is inflamed (mild localised gingivitis present)
Stage 2—the entire attached gingiva is swollen as well as inflamed (severe localised gingivitis present)
Stage 3—inflammation and swelling of the gingiva is accompanied by visible bone loss (periodontitis)
Stage 4—in addition to bone loss and the presence of severe gingivitis, bone loss is evident as well as pustular discharge and loose teeth
   The individual teeth affected, location of tartar and any specific data relating to the oral cavity of the individual dog was recorded on an ideogram of the mouth. 26 teeth were examined for each dog in total.

### Genome Screen SNP Selection

Putative SNPs for the assembly of a canine whole genome screen panel were harvested from all publicly available whole genome shotgun sequence. This data had been previously made available on-line at Genbank: (http://www.ncbi.nlm.nih.gov/projects/genome/guide/dog/). Original sequence read or putative SNP data was available for the following canine breeds:

TABLE 1
SNPs available per breed

| Breed | Approximate Sequence Count |
|---|---|
| Boxer | 34,000,000 |
| Poodle | 7,000,000 |
| German Shepherd | 100,000 |
| Rottweiler | 100,000 |
| Bedlington | 100,000 |
| Beagle | 100,000 |
| Labrador | 100,000 |
| English Shepherd | 100,000 |
| Italian Greyhound | 100,000 |
| Alaskan Malamute | 100,000 |
| Portuguese Water Dog | 100,000 |
| China Gray Wolf | 100,000 |
| Californian Coyote | 100,000 |
| Alaska Gray Wolf | 100,000 |
| Spanish Gray Wolf | 100,000 |
| Total | 42,300,000 |

Comparisons made between these traces account for 94% of the total SNPs surveyed for use in this project. The rest came from other re-sequencing projects of specific candidate genes on which the following breeds had been resequenced at a number of selected genomic loci: Labrador, Shih Tzu, Yorkshire Terrier, German Shepherd, Rottweiler, Doberman, American Cocker Spaniel, Golden Retriever and Beagle. These sources together provided approximately 2.5 million putative SNPs for use in a genome screen panel. Selection from the 2.5 million putative SNPs to the current set of 4608 used for the genome screen on the periodontal disease samples was performed using the following criteria:
Looking at quality scores at the loci in the sequence traces and excluding those with low quality scores.
Exclusion of SNPs whose flanking sequence matched in multiple locations in the genome using blast analysis. (as may indicate dispersed repeat sequences)
Priority was given to SNPs with more evidence of polymorphism (i.e. two sequence traces existed that exhibited the minor allele)
Priority was given to SNPs predicted to lie in exonic sequences.
Some SNPs that were predicted to be close to predicted genes in the transcriptome were also given priority.

SNPs from the prioritised list were selected to spread across chromosomes in order to minimize linkage disequilibrium between SNP loci. This was accomplished by choosing SNPs at regular intervals across the canine chromosomes. This resulted in the selection of a set of prioritised SNPs that were provided to Illumina, Inc. for design of multiplex SNP genotyping panels in order to perform a genome screen on the periodontal disease samples. A final set of 4608 SNPs were chosen for use in the whole genome panel, consisting of 3 sets of 1536 SNP loci. 80 ul of genomic DNA (>50 ng/ul concentration) from each of the periodontal disease samples was provided to Illumina, Inc. for genotyping against the whole genome panel or 80 μl of whole genome amplified material using a BeadArray™ technology, a fibre optic-based array system that allows miniaturised, high throughput genetic analysis. Determining the age at which periodontitis is first shown

Banfield Hospital, USA, provided information about the incident rates of periodontitis as seen by their pet centres. This data was of the dogs they had seen since 1995 that had any stage periodontitis, the ages they had first presented each stage of the disease, and their breed data. This data was then refined to an accurate description of the progression of periodontitis. First, the data was restricted to those dogs on Banfield's Optimum Wellness plans (an insurance scheme for pets that requires an in house check up every six months). This resulted in a high quality track of the progression of periodontitis in the dogs. This data was then further restricted to dogs which started on the Wellness schemes before they presented the first stage of periodontitis. This ensured that the dates that were recorded were the date (to six months) that they began to show the disease. An accurate count of the numbers of dogs that exhibit periodontitis at each age was therefore accomplished.

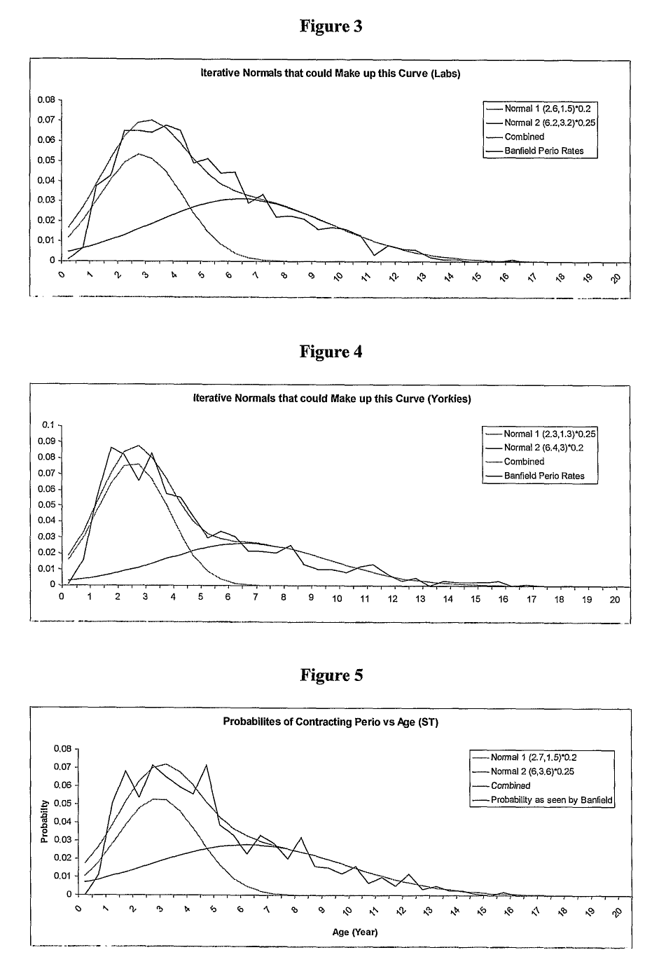

The data from Banfield also indicated the current status of the dogs; those that had the status “euthanized” or “passed on” had their closure of the wellness scheme used as a date of death, allowing an accurate life expectancy of the dog breeds to be created. This life expectancy chart was then used to calculate the expected number of dogs in the complete population (of which Banfield only sees those dogs that contract periodontitis). Combining this population with the known numbers of dogs that contract periodontitis at each age allowed the probability of dogs getting periodontitis at a certain age to be calculated. This probability does not infer anything from the current periodontitis status of the dog. The probability curves for Labrador, Shih Tzu and Yorkshire Terrier are shown in FIGS. 3 to 5.

These graphs, with their heavy bias to the younger dog, suggest the existence of three groups:
A ‘susceptible’ set, that contract periodontitis the earliest (before approximately 5 years of age)
A ‘normal’ set that can contract it throughout their entire lives.
A ‘resistant’ set, that genetically resist periodontitis (not shown on the graphs)

Each graph shows the Gaussian probability curve that describes the probability of contracting periodontitis for the susceptible set and the probability of contracting periodontitis for the normal set. Their composite is shown by another line, which has been determined iteratively, comparing the black observed probability, as calculated from Banfield's data, minimising the square error against the composite curve. These curves were entirely created from the data shared by Banfield (over 15000 dogs in these 3 breeds subject to the stringent quality control measure detailed above). The susceptible set is centred at about 2.5 years of age, the normal at about 6.2.

Determining when Dogs are Susceptible, Normal or Resistant

A dog can be labelled as susceptible to periodontitis if it develops periodontitis before a certain age. This age was determined by comparing the respective values of the probability of first displaying periodontitis if the dog is susceptible against the probability of normal periodontitis at each age. The age at which the ratio dipped below 2 was deemed to be the cut-off age for declaring susceptibility. A dog before this age with periodontitis is twice as likely to be susceptible than not. A dog was declared to be of normal periodontitis morbidity if the inverse of this ratio was greater than 2 (twice as likely to be in the normal set than otherwise). A dog was deemed to be resistant if it reached an age greater than ⅔ of the normal set's probability without contracting periodontitis.

The following ages were thus determined:

TABLE 2
Ages that define susceptibility to periodontitis

| Breed | Susceptible | Normal | Resistant |
|---|---|---|---|
| Labrador | <4 | 5.5+ | 9+ |
| Yorkshire Terrier | <3.5 | 5.5+ | 9+ |
| Shih Tzu | <3.5 | 5.5+ | 9+ |

It should be noted that between susceptible and normal lies a ˜2 year gap, where the correct assignment cannot be guaranteed. Any sample that would fall within this range was omitted, to ensure a quality data set.

The Banfield data was then used to calculate the progression of periodontitis; the delay it took for a dog to move from one stage to another. To see if there was a significant change between the susceptible and normal sets, the Banfield data was classified according to the ages above, and then evaluated. No significant difference between the sets were discovered, and it was determined that it took a year to progress between adjacent stages.

Classifying the Genotyped Dogs into Susceptible, Normal and Resistant

The progression rates of periodontitis determined from the Banfield data were then used to classify the evaluated state of the sampled dogs. The evaluating vet recorded the severity of the disease to the same scale as the Banfield data. This severity was used to alter the record of the dog's periodontitis status from “currently has periodontitis of stage X at age Y” to “most likely exhibited periodontitis stage 1 at age Z”. This would enable the modelling of the contraction age of periodontitis. These altered ages were then used to separate the dogs into “susceptible”, “normal”, or “resistant”. After this classifying process, the number of dogs in each group was counted. The Labrador data is heavily biased towards Normal and Resistant, and the Shih Tzu/Yorkies between the Susceptible and Normal.

TABLE 3
Number of dogs in each group by breed

| | | Yorkshire | |
|---|---|---|---|
| | Shih Tzu | Terrier | Labrador |
| Susceptible | 126 | 169 | 2 |
| Normal | 15 | 10 | 177 |
| Resistant | 11 | 5 | 62 |

The data was shared equally between three sets, known as 0, 1 and 2, in order to pursue the standard testing process “leave one out”. Models that are created using sets 0 and 1, and tested for their success on set 2, i.e. data it has never seen before. In this way, a consistent, and accurate, measure of the models ability is found. The data was converted into several formats, to each format allowing a slightly different analysis to be performed:
SNPs in string based formats. ‘AA’, ‘AG’, and ‘GG’ to describe the values of an AG based SNP.
SNPs in numerical format, −1, 0 and 1 denoting homozygous base 1, heterozygous, homozygous base 2 respectively
SNPs in logical format. Each SNP is represented by two columns, each containing TRUE or FALSE indicating the presence a base.

### Small Breed Model of Periodontitis

The data was analysed using GMax software, which looks for patterns of multiple interactions between gene variants that predict whether an animal will be affected. The SNPs were genotyped and converted to numbers according to their value.

TABLE 4
Conversion table for SNPs used in model

| | | | | Convert to | | |
|---|---|---|---|---|---|---|
| SNP Name | Chromosome | Coordinate | SNP | −1 | 0 | 1 |
| BICF234J8261 | 3 | 48542027 | SNP_01 | AA | AG | GG |
| BICF230J30210 | 5 | 82169224 | SNP_02 | AA | AC | CC |
| BICF231J10654 | 27 | 15328520 | SNP_03 | CC | CG | GG |
| gnl\|ti\|390102253_3 | 30 | 36202795 | SNP_04 | GG | GA | AA |
| gnl\|ti\|365340500_2 | 31 | 31752271 | SNP_05 | AA | AG | GG |
| BICF229J23852 | 34 | 22203873 | SNP_06 | AA | AG | GG |
| gnl\|ti\|351112274_1 | 36 | 33275222 | SNP_07 | CC | CG | GG |
| gnl\|ti\|351446047_1 | 11 | 51254159 | SNP_08 | AA | AG | GG |
| gnl\|dbSNP\|ss9151307 | 12 | 42104421 | SNP_09 | AA | AC | CC |
| gnl\|ti\|355608337_2 | 14 | 26734270 | SNP_10 | AA | AG | GG |
| gnl\|ti\|390142605_1 | 15 | 59978450 | SNP_11 | GG | GA | AA |
| gnl\|ti\|355640080_1 | 18 | 53445872 | SNP_12 | AA | AG | GG |

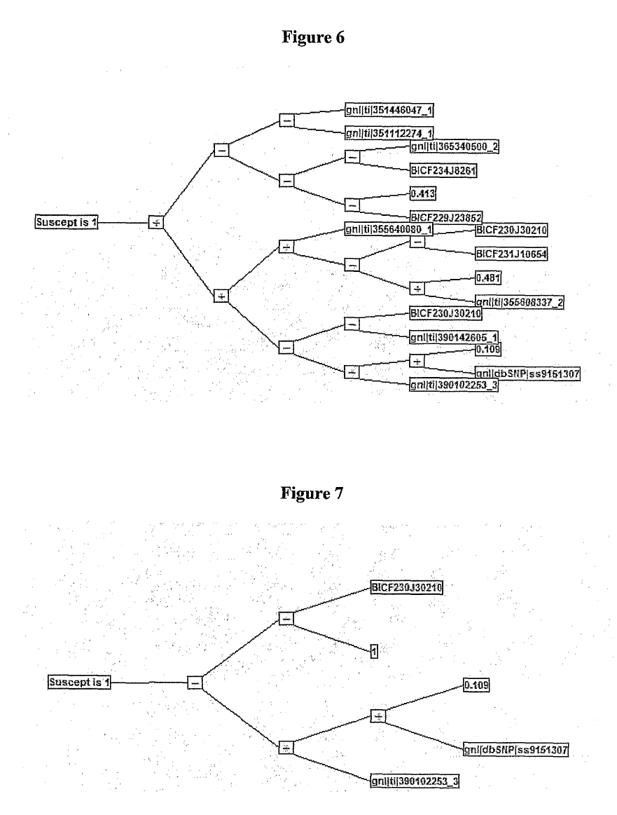

Using the data from Shih Tzus and Yorkies (two representative small dog breeds) and the numerical method of recording the genetic data (−1, 0, 1), a model was produced that could successfully determine a susceptible dog with an accuracy of 68.4% (FIG. 6). This is in the worst case, when the population at large is best represented by the test set. Over the entire data set, the accuracy rises to 87.7%.

This model (model 1) can be written in its simplest form as:

X=SNP_{—}01+2*SNP_{—}02−SNP_{—}03−SNP_{—}04−SNP_{—}05−SNP_{—}06−SNP_{—}07+SNP_{—}08−SNP_{—}09−SNP_{—}10−SNP_{—}11+SNP_{—}12−0.177

This result ‘X’ is then compared against the constant −4.176248. If X is greater than this constant, the sample is labelled as being susceptible, otherwise as non-susceptible.

The SNPs that bring the most to the model can be expressed in a far simpler model (FIG. 7). This model (model 2) has the same conversions between SNP value and numerical data, and can be expressed in its simplest form as:

X=SNP_{—}02−1.109−SNP_{—}09−SNP_{—}04

In this case, the result ‘X’ is compared against the constant −2.108751. If X is greater than or equal to this constant, the sample is labelled as susceptible, otherwise it is determined to be not susceptible. This simpler model has an accuracy of 52.6% on the test data, and an accuracy of 81.6% on the training data. This leads to a total accuracy of 71.9%.

TABLE 5
Genome screen study SNP sequences

| | Known | | In Public |
|---|---|---|---|
| SNP Name | as | Sequence | Domain? |
| BICF234J8261 | SNP_01 | 5′CTGGAGACCCAGGATTGAGTCCCACAT | Yes |
| | | CGGGTTCCCTGNATGGAGCNTGCTTCTCC | |
| | | CTCTGCCTCTGTCTCTGCTTTTCTCTCTC | |
| | | TCTCTCTCTCATGAATAAATAAATAAAAT | |
| | | CTTAAAAAAAAAAAAAAAAGAAAGAAATC | |
| | | CTAACNCCCAGTACCTCAGAATCTGACCT | |
| | | TATTTGGAGATTGGATCTTCACAGAGAT | |
| | | [A/G]ATCAAGTTAAAACGACATCATTCAG | |
| | | ACAGATCCTAATCCAAAATGANNGACATC | |
| | | TTTACAAAAAAGGAAATTTGGACATTAGA | |
| | | GATATACCTACAAGGAAAATGATGGGAAG | |
| | | AGACACCAATCATCGATGGCGTCTCCCAA | |
| | | GATGATGGGAGAAGCCAATCATCTCCAAG | |
| | | CCAAGAGAAAGACCTAGAACAGATTCTCC | |
| | | C 3′ | |
| | | (SEQ ID NO:1) | |
| BICF230J30210 | SNP_02 | 5′GTGATGTCAATGACAGCCCACAGGGCC | Yes |
| | | GTACAGTATCCATCCTGAATAACTATAAA | |
| | | TGGCAGCTCAGATTTGTCTTCATCTTTTA | |
| | | CTTCACCTCTTCCTCTAGGAAGATGTTCA | |
| | | GTACCCAATGTACTTGCCTTGGCTAACCT | |
| | | CCCCCTCGGTCTACACCTTAGGGTTTAGA | |
| | | AAGGTAGATCTCAGTGGAATCTTGCATG | |
| | | [A/C]AGCACCACTTCTTATCTTCTCTGCC | |
| | | ACCATCTATCCCAACAATTAATGTGATAA | |
| | | CCAGTTTTGTACAAAAATATTTGACAGGA | |
| | | TTTTTGCAGGCACTGTACAGTGCTTTCCC | |
| | | CTGGATGACACATCATGATTTTGGCATTT | |
| | | TCCATACAATTCTCTGGTGTTGTGAAATG | |
| | | AGATACCTGTACAAACCTGCTCAACACTG | |
| | | G 3′ | |
| | | (SEQ ID NO:2) | |
| BICF231J10654 | SNP_03 | 5′GCTACAAAAGCTCCAGGATATGCTAAA | Yes |
| | | GAAACAAAAAAGTAATAAATACAAACNAA | |
| | | TCACCTTACTTAAACCTGGACTGTACTGT | |
| | | GCTGACATGATTTTGGGTAACCAGATATT | |
| | | TAACACCTTCAAATCAGTGAGCTAAGCTG | |
| | | CCACCATTCGTCTGAATTTTTGACACTTA | |
| | | CTATTCTACAAAGCCTTGGAACTCGGGT | |
| | | [C/G]CCATGCACAATTCTTCCTTTATTCC | |
| | | TGAACACCTGCTGTTCTGTCTCCTGTGTT | |
| | | TCGATCTTTAGTNCCACTGCCCTCTTCCC | |
| | | AGTTGATCCCATCAGAAACTTTTCTGCCC | |
| | | CCTTTCTCTCTCTAATCTCCTCCATTGGA | |
| | | ATCAGTTACTGAATCAAGGAGCTCTGGCC | |
| | | TGGGTCCNTCTCACAGTCCACCCTTTCCA | |
| | | G 3′ | |
| | | (SEQ ID NO:3) | |
| gnl\|ti\|390102253_3 | SNP_04 | 5′AGGAACCAGGCCGACTCACCCAGTCTT | Yes |
| | | GACTGTGGGGCGGCCAGNGCACTGGCATT | (BICF235J53977) |
| | | TCACTGTGAAGTGTGCAGATGGGATGATA | |
| | | GAGACTTGCAAGTGGTACTTGAGTTTAGC | |
| | | CTTGAAGAATGGGTACAACCTCAAGAAGA | |
| | | GAAAGTGGAGGAAGCGGTGTTCCTCTCNT | |
| | | AGTAAACAGCAAGTGCAAAAGTATGGAA | |
| | | [C/T]GTTCAAGGACAGGGAGGAGTGATGG | |
| | | GATGTGATGAGGTGTGGGGCATGTGGTGA | |
| | | GACTGATGGGAGGTGATGCTGAGGCTGGG | |
| | | TGGGCAGAGGATTTGAGGATATGCTAAGG | |
| | | AGCGGGGTCTTCTAGAACCCCCCAGTGGA | |
| | | GATCCCATGACTTTGTTGTAGGAACATAA | |
| | | AGTTCAGACCATGAGTCAGGAGAGCAGAG | |
| | | C 3′ | |
| | | (SEQ ID NO:4) | |
| gnl\|ti\|365340500_2 | SNP_05 | 5′GGTGCTCAGTGACTGTGGATCAAGGGA | Yes |
| | | GTGCCTGAAGTGAAGCAGAGAAAGTTTTG | (BICF229J28888) |
| | | TGGCTAATTGTCTCCTCTCAGGTTGGGAT | |
| | | GGCCTGCAGCTTCTGGCAGCCACTCCCAG | |
| | | GCACCCAGCCAGTGTTTCCAGACCACGGC | |
| | | TGGGGCTGCTGTGGATGGTCCTCCAGCCA | |
| | | CGCAGTGATGAAAGGCCTCGTGAAGAAG | |
| | | [A/G]AAATGACCTTGGTGGTTGCTGGATG | |
| | | GAATGTTTCCCCTGGAGAAATCACAAAAT | |
| | | CAATCAGGTGTCCAGGCCTCAGCCTCACT | |
| | | CCTTTCCTGGGCCAGCCCAGGGTCCTGGG | |
| | | GTCAGCTCCTGGGGGGAGGTGGCTTCCTC | |
| | | TGACAGGCTAGTTTCCTTTCCAGGTCTCT | |
| | | GACTCTCCTTTGATTTTGTAATCCTGTTC | |
| | | C 3′ | |
| | | (SEQ ID NO:5) | |
| BICF229J23852 | SNP_06 | 5′AACACAGGATCCTCCAAGGGATGAGAA | Yes |
| | | AAGAAGGGGTCCTAAGGGGGTGACCCCTG | |
| | | GGCCAGAGCAGCCCCACCTGCTCAGTGGC | |
| | | TTAACAGCTTGGACTCCTCTGAGCTCCAG | |
| | | GGTTTCTCATCAGGAAAAACGAAATGAGA | |
| | | AAACTCAGGTTTACCTANAGGGCGTGGGG | |
| | | AGACTAAAATGTGATTACACACAGGGAG | |
| | | [C/T]GCCTAGCACTTCAAAGGGGCTCAGT | |
| | | AAGCCTGCCTGCCCGGCTCCCTTGCCCAC | |
| | | CTTCTACGTGGGGGGCGCGGCTGCGGAGG | |
| | | CTCTGCAGCTTCGGGCTCCTAGGGGAGGA | |
| | | ACAGGGCCGCNGGAGGCCGCCTTCTCTCC | |
| | | TTAGTGTTATTGAAAAATGTTCAAAAGAA | |
| | | ACATTTACACGAAAACAACTTTGGGAAAA | |
| | | C 3′ | |
| | | (SEQ ID NO:6) | |
| gnl\|ti\|351112274_1 | SNP_07 | 5′CTCCAGATTATAGCGTGGAGGCTACGA | Yes |
| | | CAGTTTCTGTGTCTCTGGGCTCGCTAGAA | (BICFG630J704738) |
| | | TGATGCTCGATAAAATCTGTTTCATCTAC | |
| | | NTAATAAGGCGACTCTATCCCCGAGGACT | |
| | | ATAGGGCTGTATATACGTGTCTATGAATT | |
| | | GTCGCCCAGATGCTGGAAAGCTTTATGGA | |
| | | AATTCCTGGACATTGACCGCTCTCAAGG | |
| | | [C/G]TCCCATGGTAATTTGGAGAGTGTGA | |
| | | CNCTTACTCTGCTGTCCTAGCAAATATCA | |
| | | AAGAAACGCGAGTCTGGATGAAATGTTAT | |
| | | TNACACGTGTCCCCGTCGGGTGTGAGTGT | |
| | | GGAGCAAACCAGAGGANGAGGCGTGTGCG | |
| | | TTTGCAGAAGGAAGGCANAGACGGGTGTG | |
| | | GGACATGCCCTTGGGAAAGGTGTAGATGC | |
| | | C 3′ | |
| | | (SEQ ID NO:7) | |
| gnl\|ti\|351446047_1 | SNP_08 | 5′TTTATAAATGATTATTATATATATTAA | Yes |
| | | ACATATATATACATTATATATATAATAGA | (BICF230J43570) |
| | | GTGGCATAAAATCTGAGTTTGAGTTTTAA | |
| | | GATCCTTGACCATTATAATTGCACATCCT | |
| | | CTATTTATTTCTCCAAAACTTCTACATTG | |
| | | CCCTTGGTTAGATTCCATTTTTTCTTCTT | |
| | | TAAATTGCACCCCAGGGATTATAGGCAC | |
| | | [C/T]GAATGTGATCTCATAAACTTCTAGA | |
| | | ATTATAAAATATGCATTAGCAATTGTAAC | |
| | | ACTTGGCATTATGCTAGAGTACAGAAAAT | |
| | | TTGCTATGTCCTATCCATCAACATTACCT | |
| | | CAAAGAGGGTCTGAAGAATGCAGCCCAGG | |
| | | TAATTATAAATGTTTGGAGAATCAGTAAT | |
| | | AATCATGAGAGTAGATATTAATGTATTTT | |
| | | C 3′ | |
| | | (SEQ ID NO:8) | |
| gnl\|dbSNP\|SS9151307 | SNP_09 | 5′ATGCATAAGGTTATTGCCTTAGCTCAC | Yes |
| | | TTAAAATTGCCCCCATTCAATGGTACTAT | |
| | | CAACCTTTAGTGAAGCCCTTAAAAAAACA | |
| | | AACAGGTTGAAAAAGATTAGGACAGGCAA | |
| | | ATATAGCATCTGTCTTTAGAGCTATCAAC | |
| | | TCAGGAATTCTCTCAATTATGAAATCTTG | |
| | | CAGAGAAGTTATTTTTCTTTTCCAAATT | |
| | | [A/C]TGGTGATGACAATATTCCTTACTCC | |
| | | AGAGCTGGCATTTTTACATCATCTCTGTC | |
| | | TTGTGAACCATCATCTGCTCCATCTACTT | |
| | | CTGATAAATCTGCATCCTCATCACCACTC | |
| | | CTGTTGTTCATCATCTCAGAGGAATGATC | |
| | | AAAATTAGTATGTCTTCAACTGAATCATC | |
| | | TTCCCAGTCGTTCCAATTATTATTTTTTT | |
| | | A 3′ | |
| | | (SEQ ID NO:9) | |
| gnl\|ti\|355608337_2 | SNP_10 | 5′TTGGTAATTAGACAAAAGAAGTTAAAT | Yes |
| | | TCAGTTCAGACATTTCATGCATAAGGTCC | (BICF231J51243) |
| | | TCTGACAGGCACTGGGGATTTGAAGATGT | |
| | | ATTAAACATAATAAGGGTATGCATGCAGG | |
| | | TGGTGAAGAGGGAAGAGGGANAGTTTCAG | |
| | | ATGGAGGGAATACCATGTGTAGAGAGCAT | |
| | | CGTGAGTGGAAAATATATGGGCTGTGTG | |
| | | [A/G]CGTCAGGTGAATGGCCAGCCACAGC | |
| | | TAAAGGGAATCTGTTCCTCCAAACAATAT | |
| | | AATCTAAGATTGTTGAGTGTATTGCACAA | |
| | | ACCAAGTCTCGTGTCTTATAGATGATGTT | |
| | | TGCAAATTNTAGTTAGTACACACGGATGC | |
| | | TTAAGTAAAACTCATTTGATGANGGGAAA | |
| | | GCATTTAAAGCCAAACAAAAGCAAAAATA | |
| | | T 3′ | |
| | | (SEQ ID NO:10) | |
| gnl\|ti\|390142605_1 | SNP_11 | 5′CAAAAGACTCATCTTGACTTGAATTTG | Yes |
| | | ACTTAGATGGTGAGATTTTGGACATTGAG | (BICF236J5319) |
| | | TTCTGTCTGATGAGGTTTTTGAGGGGGTC | |
| | | TTAGACAAACATTCAAAATTGAAATGCAT | |
| | | TTTGAGTTTTAAGGGATGGTTCAGTCTGG | |
| | | CAGCATTATAAGAATTTAATTGACTAAAT | |
| | | GTCTCCCCTTACTGAAAGAAGGCCAAAA | |
| | | [C/T]GTTTGCTTTGTAAGGAAGGGTCATC | |
| | | AAATATATGTGTTGGTTTTATATTATATA | |
| | | AGATTTCAATTAAGTTTTACATCATCTAT | |
| | | GTTCCTAATTTTTTTTTTATGTGGGAGGA | |
| | | ATGTGAATAACTTATTGCCACTGTGTAAA | |
| | | CCATGACTCATTTATAACAGCCTCTGAAT | |
| | | TCCTTGACTCTTCCCAGCAAAAGGTAAGG | |
| | | A 3′ | |
| | | (SEQ ID NO:11) | |
| gnl\|ti\|355640080_1 | SNP_12 | 5′CTGTGAGACCGTGGCCCAGTCAGGGCT | Yes |
| | | GCATCACTGTGCCTTAATCTTTAGGGAGG | (BICF231J46839) |
| | | GGGAGGGGTTGGGCCAGGTGCTGGGGAAG | |
| | | CTCCCGCCCCGCCTGCCTTCTGCCTCATT | |
| | | CTGACTGGCTCTGCCCTGGCAACCAGAGC | |
| | | ATCAGGTACTTACTTGGCAGGGGGAGGTG | |
| | | TCTGTGGGTCCACCCACAGCAGGTGTTT | |
| | | [A/G]GGACTAGTGCACTGGATGGAAGGAG | |
| | | AATCTCCCTTCCCAGCACCCTCTCCACGA | |
| | | CGGTAGCTGTCGCCCCCCTGTGTCGCTCT | |
| | | AGCAATACTGCAAGGACAAGTTGCCCATC | |
| | | TGCTTCTGGACCTTGAAATTCTCTTGAGC | |
| | | CTTTGCAGGGATACAGGGACCCAATCTCT | |
| | | GCTCCTCCTAGAGCCCAGTACAGGCCAAG | |
| | | C 3′ | |
| | | (SEQ ID NO:12) | |

### Methodology for Multiplex Genotyping of the 12 Predictive SNPs

The Diagnostic periodontitis SNP panel comprises 12 individual canine SNPs from different canine chromosomes. Twelve specific PCR amplicons were designed overlying these SNP positions for use in iPLEX SNP genotyping assays on the Sequenom MassARRAY system.

Each SNP was designed to be analysed twice using single base primer extension with the Sequenom iPLEX methodology. Two separate extend primers were used for each SNP, one annealing to the forward strand (f) and one to the reverse strand (r). The SNPs were configured as two separate 12-plex i-plex reactions as indicated in Table 6 (mp1 or mp2) except for the forward primer of SNP10, which cannot be multiplexed reliably into a successful assay with the other 23 markers and was therefore designed to be assayed alone in a single plex (mp3).

Multiplex PCR, product clean-up, iPLEX primer extension and subsequent de-salting were carried out according to manufacturers standard operating procedures based on Sequenom-provided user protocols. Reactions were spotted on a standard 384 well Sequenom SpectroCHIP Bioarrays and analysis of extension products performed on a Sequenom Compact MALDI-ToF mass spectrometer system according to standard operating procedures based on Sequenom-provided user protocols. Specifications of the software components and their respective versions (Assay Design 3.0; Services 2.0.8; Assay Editor 3.4.0.6; Plate Editor 3.4.0.38; TYPER Analyzer 3.4.0.18; Acquire 3.3.1.3; and Caller 3.3.1.1) are all contained within the MassARRAY Workstation package. Genotypes are scored automatically using Sequenom i-plex analysis software version 3.4.

TABLE 6

Genotyping primers and extension primers for genotyping
of 12 predictive SNPs from model using Sequenom iPLEX
technology on MASSarray system.

| | | | | |
|---|---|---|---|---|
| | Ampl | TM | | |
| SNP | Length | (NN) | PCR Primers | Extend Primer |

| | | | | | | | |
|---|---|---|---|---|---|---|---|
| SNP_1 | 102 | 47.1 | f | ACGTTGGATGGATCTGT | f | GATTGGATCTTCAC | MP2 |
| | | | | CTGAATGATGTCG | | AGAGAT | |
| | | 51.7 | r | ACGTTGGATGCAGTACC | r | TCTGAATGATGTCG | MP1 |
| | | | | TCAGAATCTGACC | | TCTTGCATG | |
| SNP_2 | 94 | 53.1 | f | ACGTTGGATGTTGTTGG | f | AGATCTCAGTGGAA | MP1 |
| | | | | GATAGATGGTGGC | | TCTTGCATG | |
| | | 45.8 | r | ACGTTGGATGAGAAAGG | r | AAGATAAGAAGTGG | MP2 |
| | | | | TAGATCTCAGTGG | | TGCT | |
| SNP_3 | 119 | 54.4 | f | ACGTTGGATGCAGGTGT | f | AAGCCTTGGAACTC | MP2 |
| | | | | TCAGGAATAAAGG | | GGGT | |
| | | 48.0 | r | ACGTTGGATGAAGCTGC | r | AGGAAGAATTGTGC | MP2 |
| | | | | CACCATTCGTCTG | | ATGG | |
| SNP_4 | 115 | 46.0 | f | ACGTTGGATGAAGAGAA | f | CTCCCTGTCCTTGA | MP2 |
| | | | | AGTGGAGGAAGCG | | AC | |
| | | 51.3 | r | ACGTTGGATGATCACAT | r | AGCAAGTGCAAAAG | MP1 |
| | | | | CCCATCACTCCTC | | TATGGAA | |
| SNP_5 | 86 | 48.4 | f | ACGTTGGATGACATTCC | f | AGGCCTCGTGAAGT | MP2 |
| | | | | ATCCAGCAACCAC | | T | |
| | | 45.5 | r | ACGTTGGATGCAGCCAC | r | ACCACCAAGGTCAT | MP1 |
| | | | | GCAGTGATGAAAG | | TT | |
| SNP_6 | 84 | 56.4 | f | ACGTTGGATGCGTGGGG | f | GCCCCTTTGAAGTG | MP2 |
| | | | | AGACTAAAATGTG | | CTAGGC | |
| | | 45.5 | r | ACGTTGGATGCTTACTG | r | TGATTACACACAGG | MP1 |
| | | | | AGCCCCTTTGAAG | | GAG | |
| SNP_7 | 96 | 45.2 | f | ACGTTGGATGAATTCCT | f | TCCAAATTACCATG | MP2 |
| | | | | GGACATTGACCGC | | GGA | |
| | | 57.4 | r | ACGTTGGATGTGCTAGG | r | TGGACATTGACCGC | MP1 |
| | | | | ACAGCAGAGTAAG | | TCTCAAGG | |
| SNP_8 | 126 | 46.2 | f | ACGTTGGATGTCTTTAA | f | AGAAGTTTATGAGA | MP2 |
| | | | | ATTGCACCCCAGG | | TCACATTC | |
| | | 52.2 | r | ACGTTGGATGCTCTAGC | r | CCCCAGGGATTATA | MP1 |
| | | | | ATAATGCCAAGTG | | GGCAC | |
| SNP_9 | 121 | 45.2 | f | ACGTTGGATGAATGCCA | f | GAAGTTATTTTTCT | MP2 |
| | | | | GCTCTGGAGTAAG | | TTTCCAAATT | |
| | | 50.6 | r | ACGTTGGATGATCAACT | r | GAGTAAGGAATATT | MP1 |
| | | | | CAGGAATTCTCTC | | GTCATCACCA | |
| SNP_10 | 100 | 46.7 | f | ACGTTGGATGATTCCCT | f | GAAAATATATGGGC | MP3 |
| | | | | TTAGCTGTGGCTG | | TGTGTG | |
| | | | r | ACGTTGGATGCATGTGT | r | TAGCTGTGGCTGGC | MP1 |
| | | | | AGAGAGCATCGTG | | GCCATTCACCTGAC | |
| | | | | | | G | |
| SNP_11 | 86 | 46.9 | f | ACGTTGGATGGACTAAA | f | CCTTCCTTACAAAG | MP2 |
| | | | | TGTCTCCCCTTAC | | CAAAC | |
| | | 48.1 | r | ACGTTGGATGCATATAT | r | TTACTGAAAGAAGG | MP1 |
| | | | | TTGATGACCCTTCC | | CCAAAA | |
| SNP_12 | 105 | 49.8 | f | ACGTTGGATGATTCTCC | f | CCCACAGCAGGTGT | MP2 |
| | | | | TTCCATCCAGTGC | | TT | |
| | | 49.7 | r | ACGTTGGATGTCAGGTA | r | CATCCAGTGCACTA | MP1 |
| | | | | CTTACTTGGCAGG | | GTCC | |

### EXAMPLE 2

### Testing the Model on Further Dog Samples

Model 1 was tested on further dog samples that became available and were appropriate for testing on. The test was performed on 17 Yorkshire Terrier, and 3 Shih Tzu dog samples. These samples comprised samples of dogs that since Example 1 was carried out, had subsequently developed periodontitis. The samples used were from dogs that were younger than 3.5 years of age, so that it could be reasonably certain that the dogs could be classed as susceptible. Other samples comprised samples from dogs that did not have periodontitis when Example 1 was carried out, and still do not. These samples were from dogs that were older than 5 years (with an age difference of more than 1 year).

Using this data 15/17 Yorkshire Terriers were correctly called (1 false positive, 1 false negative, 10 positive, 5 negative), and ⅔ Shih Tzus correctly called (1 false negative, 2 positive, no resistant).

The model thus had an accuracy of 85% in these reevaluated dogs.

### EXAMPLE 3

### Determining Breeds that are Genetically Related to Yorkshire Terrier

The twelve SNP loci [SNP_{—}01 to SNP_{—}12] that were optimally typed to determine whether an individual dog is susceptible to early-onset periodontal disease were genotyped across over 4410 reference canine samples from over 130 canine breeds (signified by Kennel club pedigree data for most of the dogs). Only purebred samples were used. The number of samples used for each breed are set out in Table 7 in Example 4.

Nine of the twelve loci [SNP_{—}01, SNP_{—}02, SNP_{—}03, SNP_{—}04, SNP_{—}05, SNP_{—}06, SNP_{—}07, SNP_{—}08, SNP_{—}10] were typed in a whole genome screen experiment with 1,536 selected SNP loci, across the canine genome. This was accomplished by choosing a sub-set of specific SNPs across the canine chromosomes from the successfully genotyped SNP loci from the 4,608 genome wide marker set typed in Example 1. This selection resulted in the selection of a set of prioritised SNP set that were provided to Illumina, Inc. for design of multiplex SNP genotyping panels in order to perform a genome screen on the set of reference breed samples from multiple canine breeds.

80 μl of genomic DNA (>50 ng/μl concentration) from each of the 4410 reference breed samples was provided to Illumina, Inc. for genotyping against the 1536 SNP whole genome panel using the BeadArray™ technology, a fibre optic-based array system that allows miniaturised, high throughput discriminatory genetic analysis. These markers were typed against the 4410 reference dogs and the data returned by ftp download from a secure ftp site.

The other three SNP loci [SNP_{—}09, SNP_{—}11 and SNP_{—}12] were genotyped separately using the Sequenom i-plex technology as a custom research project by GeneSeek, LLC. Each SNP genotype was genotyped in both forward and reverse directions to obtain reliable consensus calls for each SNP locus. This analysis was performed to ascertain the genotypes at each of the same 4410 reference canine breed as samples typed in the canine genome screen described above. Genotype data for each of the loci was returned as a comma-separated variable (.csv) file from GeneSeek, LLC.

The genotype information for each of the 12 SNPs was used to create a genetic distance matrix. The genetic distance matrix enabled a comparison of how genetically similar each of the 130 different breeds are to each other. A metric value representing the degree of relatedness between 2 breeds was calculated by determining the difference in allele frequency for each of the 12 SNPs between those 2 breeds. Allelic frequency for each SNP was determined by calculating the frequency of one of the SNP alleles across each sample of the breed of interest. Once the difference in allelic frequency of each of the 12 SNPs had been calculated for the 2 breeds that were being compared, these frequencies were squared and then summed. The resulting metric value provided an indication of how genetically related the two breeds were. A value of 0.4 or less indicates that the 2 breeds are significantly related to each other across the 12 SNPs. A metric value was calculated for every possible combination of pairs of breeds out of the 130 breeds.

The genetic distance matrix was used to find which of the 130 breeds are sufficiently related to Yorkshire Terrier dogs across the 12 SNPs. The matrix shown in FIG. 9 shows the breeds that are most related genetically to Yorkshire Terrier dogs across the 12 SNP positions. A metric value of 0.4 or less was deemed to indicate sufficient genetic relatedness. These values are highlighted in the FIG. 9. The breeds shown in FIG. 9 are therefore sufficiently genetically related to Yorkshire Terriers across the 12 SNPs to enable the periodontitis susceptibility test based on these 12 SNPs to work in these breeds.

### EXAMPLE 4

### In Silico Typing of Dogs from Other Canine Breeds

Data analysis was performed for each of the 4410 canine reference samples (detailed in Example 3) to obtain an in silico predictive consensus call for the periodontal disease status for each dog given the observed and empirically derived composite genotypes at each of the 12 SNP loci of the SNPs that comprise the optimal periodontal disease susceptibility determining set.

The test was only applied to samples that were from purebred dogs, and of those, only those where genotyping succeeded in all twelve SNPs. Only breeds with ten or more samples were deemed to have enough depth to test. With more than 10 samples in a breed, it is a reasonable expectation for at least one dog to be, in reality, susceptible, and at least one to be non-susceptible. Thus with more than 10 dogs, the test should be able to pick out at least one in either category. In order for the test results to have meaning within a breed, the results had to have at least one in either category.

The consensus genotype call for each locus (Illumina bead station or Sequenom i-plex technology) was used as a consensus data set to be applied to the predictive model. A call for each dog was recorded (susceptible or non-susceptible) based on the composite pattern of genotypes observed at each locus having been applied to the periodontitis susceptibility defining models.

Of the breeds the genetic relatedness matrix deemed closely related enough to Yorkshire Terriers (Example 3; FIG. 9), every breed was found to have at least one suspected susceptibility dog using the 12 SNP model (model 1). This is shown in Table 7 below. The results suggest that the test is capable of working in these breeds. Only one breed; Dachshund (Short Haired), did not have 10 samples, it does however, exhibit the ability to split samples between the classifications. This can be seen in Table 7.

Table 8 demonstrates the use of the 3 SNP model (model 2) in the breeds that are genetically related to Yorkshire Terriers. This model is not as powerful as the 12 SNP model. This is demonstrated by the fact that for some of the breeds, susceptibility to periodontitis is called for every sample of the breed (100% susceptibility ratio). As the 3 SNP model contains the 3 most informative SNP loci out of the 12 SNPs, this model can be used as a preliminary screen for periodontitis susceptibility. A more accurate screen involving more SNP loci can then be applied to dogs highlighted as susceptible.

TABLE 7
Testing the ability of the 12 SNP model on other “close” breeds

| | Number | | | Suscep- |
|---|---|---|---|---|
| Breeds close to | of | Called | Called Non- | tible |
| Yorkshire Terriers | Samples | Susceptible | Susceptible | Ratio |
| Terrier, Yorkshire | 25 | 11 | 14 | 44% |
| Pomeranian | 27 | 13 | 14 | 48% |
| Terrier, toy fox | 19 | 12 | 7 | 63% |
| Terrier, Silky | 12 | 7 | 5 | 58% |
| Briard | 20 | 10 | 10 | 50% |
| Poodle, Miniature | 22 | 14 | 8 | 64% |
| Pointer, German | 27 | 21 | 6 | 78% |
| Shorthaired | | | | |
| Terrier, Jack Russell | 27 | 19 | 8 | 70% |
| Australian Cattle Dog | 20 | 14 | 6 | 70% |
| Spaniel, American | 34 | 27 | 7 | 79% |
| Cocker | | | | |
| American Eskimo | 21 | 15 | 6 | 71% |
| Terrier, Australian | 20 | 11 | 9 | 55% |
| Terrier, Cairn | 28 | 25 | 3 | 89% |
| Dalmatian | 28 | 4 | 24 | 14% |
| Spaniel, Sussex | 21 | 6 | 15 | 29% |
| Spaniel, English Cocker | 23 | 17 | 6 | 74% |
| Poodle, Standard | 16 | 7 | 9 | 44% |
| Bichon Frise | 26 | 21 | 5 | 81% |
| Belgian Malinois | 11 | 5 | 6 | 45% |
| Poodle | 19 | 16 | 3 | 84% |
| Dachshund, Short | 6 | 3 | 3 | 50% |
| Haired | | | | |
| Dachshund | 21 | 17 | 4 | 81% |
| Havanese | 17 | 15 | 2 | 88% |
| Terrier, Welsh | 23 | 8 | 15 | 35% |
| Retriever, Labrador | 35 | 18 | 17 | 51% |
| Whippet | 31 | 15 | 16 | 48% |
| Beagle | 25 | 15 | 10 | 60% |
| Chihuahua | 20 | 16 | 4 | 80% |
| Norwegian Elkhound | 25 | 17 | 8 | 68% |
| Schnauzer, Miniature | 25 | 9 | 16 | 36% |
| Schnauzer, Standard | 20 | 19 | 1 | 95% |
| Shepherd, Australian | 17 | 12 | 5 | 71% |
| Spaniel, American | 22 | 20 | 2 | 91% |
| Water | | | | |
| Terrier, Staffordshire | 19 | 14 | 5 | 74% |
| Bull | | | | |
| Spaniel, Tibetan | 26 | 6 | 20 | 23% |
| Keeshond | 12 | 7 | 5 | 58% |
| Viszla | 25 | 21 | 4 | 84% |
| Terrier, Boston | 25 | 21 | 4 | 84% |

TABLE 8
Testing the ability of the 3 SNP model on other “close” breeds

| | Number | Called | | Suscep- |
|---|---|---|---|---|
| Breeds Closest to Yorkshire | Of | Suscep- | Called Non | tible |
| Terriers | Samples | tible | Susceptible | Ratio |
| Yorkshire Terrier | 25 | 23 | 2 | 92% |
| Pomeranian | 28 | 25 | 3 | 89% |
| Fox Terrier (Toy) | 19 | 15 | 4 | 79% |
| Silky Terrier | 12 | 10 | 2 | 83% |
| Briard | 20 | 15 | 5 | 75% |
| Poodle (small) | 26 | 25 | 1 | 96% |
| German Shorthaired | 29 | 28 | 1 | 97% |
| Pointer | | | | |
| Jack Russell Terrier | 27 | 25 | 2 | 93% |
| Australian Cattle Dog | 21 | 20 | 1 | 95% |
| American Cocker | 34 | 20 | 14 | 59% |
| Spaniel | | | | |
| American Eskimo Dog | 21 | 21 | 0 | 100% |
| Australian Terrier | 20 | 12 | 8 | 60% |
| Cairn Terrier | 28 | 19 | 9 | 68% |
| Dalmatian | 28 | 21 | 7 | 75% |
| Sussex Spaniel | 21 | 21 | 0 | 100% |
| English Cocker Spaniel | 23 | 13 | 10 | 57% |
| Poodle (Standard) | 19 | 19 | 0 | 100% |
| Bichon Frise | 26 | 26 | 0 | 100% |
| Belgian Malinois | 11 | 11 | 0 | 100% |
| Poodle | 20 | 20 | 0 | 100% |
| Dachshund (Short | 6 | 6 | 0 | 100% |
| Haired) | | | | |
| Dachshund | 36 | 27 | 9 | 75% |
| Havanese | 17 | 14 | 3 | 82% |
| Welsh Terrier | 24 | 20 | 4 | 83% |
| Labrador Retriever | 36 | 24 | 12 | 67% |
| Whippet | 32 | 26 | 6 | 81% |
| Beagle | 26 | 24 | 2 | 92% |
| Chihuahua | 20 | 16 | 4 | 80% |
| Norwegian Elkhound | 26 | 13 | 13 | 50% |
| Schnauzer (Miniature) | 27 | 23 | 4 | 85% |
| Schnauzer (Standard) | 20 | 19 | 1 | 95% |
| Australian Shepherd | 17 | 15 | 2 | 88% |
| Dog | | | | |
| American Water | 22 | 16 | 6 | 73% |
| Spaniel | | | | |
| Staffordshire Bull | 19 | 19 | 0 | 100% |
| Terrier | | | | |
| Tibetan Spaniel | 28 | 12 | 16 | 43% |
| Keeshond | 13 | 11 | 2 | 85% |
| Hungarian Viszla | 26 | 25 | 1 | 96% |
| Boston Terrier | 27 | 17 | 10 | 63% |

1. A method for determining susceptibility to periodontitis in a Shih Tzu dog, Yorkshire Terrier dog or a dog of a breed that is genetically related to the Shih Tzu or Yorkshire Terrier breed, the method comprising:
a) typing the nucleotide present in the genome of the dog at or at a position equivalent to each of the following:
position 201 of SEQ ID NO: 2 (SNP_{—}02), or a position that is in linkage disequilibrium with this position,
position 201 of SEQ ID NO: 4 (SNP_{—}04), or a position that is in linkage disequilibrium with this position, and
position 201 of SEQ ID NO: 9 (SNP_{—}09), or a position that is in linkage disequilibrium with this position, and
b) thereby determining whether the dog is susceptible to periodontitis.

2. A method according to claim 1, wherein said typing is carried out on a sample taken from the dog before the dog is about 2 years old.

3. A method according to claim 1, which further comprises typing the nucleotide present in the genome of the dog at or at a position equivalent to each of the following:
position 201 in SEQ ID NO:1 (SNP_{—}01), or a position that is in linkage disequilibrium with this position,
position 201 in SEQ ID NO:3 (SNP_{—}03), or a position that is in linkage disequilibrium with this position,
position 201 in SEQ ID NO:5 (SNP_{—}05), or a position that is in linkage disequilibrium with this position,
position 201 in SEQ ID NO:6 (SNP_{—}06), or a position that is in linkage disequilibrium with this position,
position 201 in SEQ ID NO:7 (SNP_{—}07), or a position that is in linkage disequilibrium with this position,
position 201 in SEQ ID NO:8 (SNP_{—}08), or a position that is in linkage disequilibrium with this position,
position 201 in SEQ ID NO:10 (SNP_{—}10), or a position that is in linkage disequilibrium with this position,
position 201 in SEQ ID NO: 11 (SNP_{—}11), or a position that is in linkage disequilibrium with this position, and
position 201 in SEQ ID NO: 12 (SNP_{—}12), or a position that is in linkage disequilibrium with this position.

4. A method according to claim 1, wherein the dog is a Shih Tzu or Yorkshire Terrier.

5. A method according to claim 1, wherein the dog is a toy dog and is selected from Yorkshire Terrier, Pomeranian, Toy Fox Terrier, Silky Terrier, Bichon Frise, Havanese and Chihuahua; or is a Terrier and is selected from Jack Russell Terrier, Australian Terrier, Cairn Terrier, Welsh Terrier and Staffordshire Bull Terrier; or is a pastoral dog (herding group) and is selected from Briard, Australian Cattle Dog, Belgian Malinois and Australian Shepherd; or is a utility dog (non-sporting) and is selected from Miniature Poodle, American Eskimo, Dalmatian, Standard Poodle, Poodle, Miniature Schnauzer, Standard Schnauzer, Tibetan Spaniel, Keeshond and Boston Terrier; or is a gun dog (sporting) and is selected from German Short Haired Pointer, American Cocker Spaniel, Sussex Spaniel, English Cocker Spaniel, Labrador Retriever, American Water Spaniel and Viszla; or is a hound and is selected from Short Haired Dachshund, Dachshund, Whippet, Beagle and Norwegian Elkhound.

6. (canceled)

7. (canceled)

8. (canceled)

9. (canceled)

10. (canceled)

11. A method for determining susceptibility to periodontitis in a dog, the method comprising:
a) typing the nucleotide present in the genome of the dog at or at a position equivalent to one or more of the following:
position 201 of SEQ ID NO: 1 (SNP_{—}01), or a position that is in linkage disequilibrium with this position,
position 201 of SEQ ID NO: 2 (SNP_{—}02), or a position that is in linkage disequilibrium with this position,
position 201 of SEQ ID NO: 3 (SNP_{—}03), or a position that is in linkage disequilibrium with this position,
position 201 of SEQ ID NO: 4 (SNP_{—}04), or a position that is in linkage disequilibrium with this position,
position 201 of SEQ ID NO: 5 (SNP_{—}05), or a position that is in linkage disequilibrium with this position,
position 201 of SEQ ID NO: 6 (SNP_{—}06), or a position that is in linkage disequilibrium with this position,
position 201 of SEQ ID NO: 7 (SNP_{—}07), or a position that is in linkage disequilibrium with this position,
position 201 of SEQ ID NO: 8 (SNP_{—}08), or a position that is in linkage disequilibrium with this position,
position 201 of SEQ ID NO: 9 (SNP_{—}09), or a position that is in linkage disequilibrium with this position,
position 201 of SEQ ID NO: 10 (SNP_{—}10), or a position that is in linkage disequilibrium with this position,
position 201 of SEQ ID NO: 11 (SNP_{—}11), or a position that is in linkage disequilibrium with this position, or
position 201 of SEQ ID NO: 12 (SNP_{—}12), or a position that is in linkage disequilibrium with this position; and
(b) thereby determining whether the dog is susceptible to periodontitis.

12. A method according to claim 11, wherein at least 3 SNP positions are typed.

13. A method according to claim 1, wherein the dog is a Shih Tzu or Yorkshire Terrier or is a breed that is genetically related to a Shih Tzu or to a Yorkshire Terrier breed.

14. A method according to claim 11, wherein step (a) comprises contacting a polynucleotide or protein in a sample from the dog with a specific binding agent and determining whether the agent binds to the polynucleotide or protein.

15. A method according to claim 14, wherein step
(a) comprises contacting a polynucleotide in a sample from the dog with a specific binding agent and wherein the specific binding agent is a polynucleotide.

16. A method according to claim 1, wherein the nucleotide present at a polymorphic position is detected by measuring the mobility of a polynucleotide during gel electrophoresis.

17. A method of preparing customised food for a dog that is susceptible to periodontitis, the method comprising:
a) determining whether the dog is susceptible to periodontitis by a method according to claim 11; and
b) preparing food suitable for the dog.

18. A method according to claim 17, wherein the customised dog food comprises ingredients that prevent or alleviate periodontitis, and/or does not include ingredients that contribute to or aggravate periodontitis.

19. A method according to claim 18, wherein the customised dog food comprises antimicrobial natural oils, zinc, polyphosphates (STPP), furanones, green tea, borage oil, omega 3 fatty acids, vitamin E, aloe vera, co-enzyme Q10, vitamin C or folic acid.

20. A method according to claim 17, further comprising providing the food to the dog, the dog's owner or the person responsible for feeding the dog.

21. A method of providing a customised dog food, the method comprising providing food suitable for a dog that is susceptible to periodontitis to the dog, the dog's owner or the person responsible for feeding the dog, wherein the dog has been genetically determined to be susceptible to periodontitis by a method according to claim 11.

22. (canceled)

23. A method of treating a dog for periodontitis, the method comprising administering to the dog an effective amount of a therapeutic compound which prevents or treats periodontitis, wherein the dog has been identified as being susceptible to periodontitis by a method according to claim 11.

24. A method of providing care recommendations for a dog, the method comprising:
a) determining whether the dog is susceptible to periodontitis by a method according to claim 11; and
b) providing appropriate care recommendations to the dog's owner or carrier.

25. A method according to claim 24, wherein the care recommendations comprise instructions for tooth brushing and/or to carry out a scale and root plane on a regular basis.

26. A database comprising information relating to polymorphisms in the canine genome and their association with periodontitis.

27. A method for determining susceptibility to periodontitis in a dog, the method comprising:
(a) inputting data of the nucleotide present at one or more polymorphic positions in the dog's genome to a computer system;
(b) comparing the data to a computer database, which database comprises information relating to canine genomic polymorphisms as defined in claims 1, 3 or 6 and their association with periodontitis; and
(c) determining on the basis of the comparison whether the dog is susceptible to periodontitis.

28. (canceled)

29. A computer program encoded on a computer-readable medium and comprising program code which, when executed, performs all the steps of claim 27, or a computer system arranged to perform a method according to claim 27 comprising:
(a) means for receiving data of the nucleotide present at one or more polymorphic positions in the dog's genome;
(b) a module for comparing the data with a database comprising information relating to canine genomic polymorphisms as defined in claims 1, 3 or 6 and their association with susceptibility to periodontitis; and
(c) means for determining on the basis of said comparison whether the dog is susceptible to periodontitis.

30. (canceled)

31. (canceled)

32. (canceled)

33. (canceled)

34. A kit for carrying out the method of claim 11, comprising a probe or primer that is capable of detecting a polymorphism as defined in any one of claims 1, 3 or 11.
